SEQUENCE LISTING (54 sequences)

SEQ ID NO: 1 - protein, 282 aa, Artificial Sequence [sequence omitted]

SEQ ID NO: 2 - protein, 302 aa, Artificial Sequence
  DNASE1-LIKE 1 (NP_006721.1)
MHYPTALLFLILANGAQAFRICAFNAQRLTLAKVAREQVMDTLVRILARCDIMVLQEVVDSSGSAIPLLLRELNRFDGSGPYSTLSSPQLGRSTYMETYVYFYRSHKTQVLSSYVYNDEDDVFAREPFVAQFSLPSNVLPSLVLVPLHTTPKAVEKELNALYDVFLEVSQHWQSKDVILLGDFNADCASLTKKRLDKLELRTEPGFHWVIADGEDTTVRASTHCTYDRVVLHGERCRSLLHTAAAFDFPTSFQLTEEEALNISDHYPVEVELKLSQAHSVQPLSLTVLLLLSLLSPQLCPAA

SEQ ID NO: 3 - protein, 299 aa, Artificial Sequence
  DNASE1-LIKE 2 (NP_001365.1)
MGGPRALLAALWALEAAGTAALRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 4 - protein, 305 aa, Artificial Sequence
  DNASE1-LIKE 3; Isoform 1 (NP_004935.1)
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 5 - protein, 275 aa, Artificial Sequence
  DNASE1-LIKE 3, Isoform 2 (NP_001243489.1)
MSRELAPLLLLLLSIHSALAMRICSFNVRSFGESKQEDKNAMDVIVKVIKRCDIILVMEIKDSNNRICPILMEKLNREKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 6 - protein, 360 aa, Artificial Sequence
  DNASE2A (O00115)
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 7 - protein, 361 aa, Artificial Sequence
  DNASE2B (Q8WZ79)
MKQKMMARLLRTSFALLFLGLFGVLGAATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 8 - protein, 304 aa, Artificial Sequence
  D1L3 + D1-BB#50
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTATPTNCAYDRIVLRGQEIVSSVVPKSNSVFDFQKAYKLTEEEALDVSDHFPVEFKLQSSRAFTNSKKSVTLRKKTKSKRS

SEQ ID NO: 9 - protein, 305 aa, Artificial Sequence
  D1L3 + D1-BB#51
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTHCAYDRIVLRGQEIVSSVVPKSNSVFDFQKAYKLTEEEALDVSDHFPVEFKLQSSRAFTNSKKSVTLRKKTKSKRS

SEQ ID NO: 10 - protein, 305 aa, Artificial Sequence
  D1L3 + D1-BB#52
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVVAGQEIVSSVVPKSNSVFDFQKAYKLTEEEALDVSDHFPVEFKLQSSRAFTNSKKSVTLRKKTKSKRS

SEQ ID NO: 11 - protein, 305 aa, Artificial Sequence
  D1L3 + D1-BB#53
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGMLLRGAVVPKSNSVFDFQKAYKLTEEEALDVSDHFPVEFKLQSSRAFTNSKKSVTLRKKTKSKRS

SEQ ID NO: 12 - protein, 305 aa, Artificial Sequence
  D1L3 + D1-BB#54
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPDSNSVFDFQKAYKLTEEEALDVSDHFPVEFKLQSSRAFTNSKKSVTLRKKTKSKRS

SEQ ID NO: 13 - protein, 305 aa, Artificial Sequence
  D1L3 + D1-BB#55
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNALPDFQKAYKLTEEEALDVSDHFPVEFKLQSSRAFTNSKKSVTLRKKTKSKRS

SEQ ID NO: 14 - protein, 305 aa, Artificial Sequence
  D1L3 + D1-BB#56
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFNFQKAYKLTEEEALDVSDHFPVEFKLQSSRAFTNSKKSVTLRKKTKSKRS

SEQ ID NO: 15 - protein, 305 aa, Artificial Sequence
  D1L3 + D1-BB#57
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQAAYKLTEEEALDVSDHFPVEFKLQSSRAFTNSKKSVTLRKKTKSKRS

SEQ ID NO: 16 - protein, 305 aa, Artificial Sequence
  D1L3 + D1-BB#58
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYGLTEEEALDVSDHFPVEFKLQSSRAFTNSKKSVTLRKKTKSKRS

SEQ ID NO: 17 - protein, 305 aa, Artificial Sequence
  D1L3 + D1-BB#59
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLSDQLALDVSDHFPVEFKLQSSRAFTNSKKSVTLRKKTKSKRS

SEQ ID NO: 18 - protein, 305 aa, Artificial Sequence
  D1L3 + D1-BB#60
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEAQAISDHFPVEFKLQSSRAFTNSKKSVTLRKKTKSKRS

SEQ ID NO: 19 - protein, 305 aa, Artificial Sequence
  D1L3 + D1-BB#61
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHYPVEFKLQSSRAFTNSKKSVTLRKKTKSKRS

SEQ ID NO: 20 - protein, 305 aa, Artificial Sequence
  D1L3 + D1-BB#62
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEVMLQSSRAFTNSKKSVTLRKKTKSKRS

SEQ ID NO: 21 - protein, 282 aa, Artificial Sequence
  D1L3 + D1-BB#63
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLK

SEQ ID NO: 22 - protein, 304 aa, Artificial Sequence
  S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKR

SEQ ID NO: 23 - protein, 302 aa, Artificial Sequence
  K303_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKS

SEQ ID NO: 24 - protein, 293 aa, Artificial Sequence
  V294_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKS

SEQ ID NO: 25 - protein, 290 aa, Artificial Sequence
  K291_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNS

SEQ ID NO: 26 - protein, 284 aa, Artificial Sequence
  R285_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSS

SEQ ID NO: 27 - protein, 282 aa, Artificial Sequence
  S283_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQ

SEQ ID NO: 28 - protein, 81 aa, Artificial Sequence
  Alpha mating factor (P01149)
MRFPSIFTAVLFAASSALAAPVNTTTEDETAQIPAEAVIGYSDLEGDFDVAVLPFSNSTNNGLLFINTTIASIAAKEEGVS

SEQ ID NO: 29 - protein, 24 aa, Artificial Sequence
  Human Albumin Secretory Signal Peptide + Propeptide (P02768)
MKWVTFISLLFLFSSAYSRGVFRR

SEQ ID NO: 30 - protein, 20 aa, Artificial Sequence
  Human DNASE1L3 Signal Peptide (Q13609)
MSRELAPLLLLLLSIHSALA

SEQ ID NO: 31 - protein, 5 aa, Artificial Sequence
  Synthetic Sequence
GGGGS

SEQ ID NO: 32 - protein, 15 aa, Artificial Sequence
  Synthetic Sequence
GGGGSGGGGSGGGGS

SEQ ID NO: 33 - protein, 14 aa, Artificial Sequence
  Synthetic Sequence
APAPAPAPAPAPAP

SEQ ID NO: 34 - protein, 12 aa, Artificial Sequence
  Synthetic Sequence
AEAAAKEAAAKA

SEQ ID NO: 35 - protein, 7 aa, Artificial Sequence
  Synthetic Sequence
SGGSGSS

SEQ ID NO: 36 - protein, 16 aa, Artificial Sequence
  Synthetic Sequence
SGGSGGSGGSGGSGSS

SEQ ID NO: 37 - protein, 31 aa, Artificial Sequence
  Synthetic Sequence
SGGSGGSGGSGGSGGSGGSGGSGGSGGSGSS

SEQ ID NO: 38 - protein, 29 aa, Artificial Sequence
  Synthetic Sequence
GGSGGSGGSGGSGGSGGSGGSGGSGGSGS

SEQ ID NO: 39 - protein, 585 aa, Artificial Sequence [sequence omitted]

SEQ ID NO: 40 - protein, 625 aa, Artificial Sequence
  Human Factor XI
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 41 - protein, 638 aa, Artificial Sequence
  Human prekallikrein
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 42 - protein, 11 aa, Artificial Sequence
  DNase epitope
MGDFNAGCSYV

SEQ ID NO: 43 - protein, 25 aa, Artificial Sequence
  FXIIa-susceptible linker (Factor XI peptide)
CTTKIKPRIVGGTASVRGEWPWQVT

SEQ ID NO: 44 - protein, 13 aa, Artificial Sequence
  FXIIa-susceptible linker
GGGGSPRIGGGGS

SEQ ID NO: 45 - protein, 26 aa, Artificial Sequence
  FXIIa-susceptible linker (Prekallikrein peptide)
VCTTKTSTRIVGGTNSSWGEWPWQVS

SEQ ID NO: 46 - protein, 7 aa, Artificial Sequence
  FXIIa-susceptible linker (Prekallikrein peptide)
STRIVGG

SEQ ID NO: 47 - protein, 878 aa, Artificial Sequence
  Albumin, DNASE1L3 Variant
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGSGGSGGSGGSGGSGGSGGSGGSGGSGSSMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQ

SEQ ID NO: 48 - protein, 885 aa, Artificial Sequence
  Albumin, DNASE1L3
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 49 - protein, 560 aa, Artificial Sequence
  DNASE1L3, Fc Fragment
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSSGGSGGSGGSGGSGGSGGSGGSGGSGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSVSVMHEALHNHYTQKSLSLSPGK

SEQ ID NO: 50 - protein, 538 aa, Artificial Sequence
  DNASE1L3, Fc Fragment
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSGGSGGSGGSGGSGGSGGSGGSGGSGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSVSVMHEALHNHYTQKSLSLSPGK

SEQ ID NO: 51 - protein, 297 aa, Artificial Sequence
  K298_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLR

SEQ ID NO: 52 - protein, 296 aa, Artificial Sequence
  R297_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTL

SEQ ID NO: 53 - protein, 292 aa, Artificial Sequence
  S293_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKK

SEQ ID NO: 54 - protein, 291 aa, Artificial Sequence
  K292_S305del
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSK

(19) United States
(12) Patent Application Publication
(10) Pub. No.: US 2023/0072226 A1
(43) Pub. Date: Mar. 9, 2023

(54) BASIC DOMAIN-DELETED DNASE1-LIKE 3 AND USES THEREOF

(71) Applicant: NEUTROLIS, INC., Cambridge, MA (US)

(72) Inventor: Tobias A. FOX, Wellesley, MA (US)

(21) Appl. No.: 17/800,950

(22) PCT Filed: Feb. 22, 2021
(86) PCT No.: PCT/US2021/019048

§ 371 Date: Aug. 19, 2022

Related U.S. Application Data
(60) Provisional application No. 63/064,566, filed on Aug. 12, 2020.
(60) Provisional application No. 63/040,742, filed on Jun. 18, 2020.
(60) Provisional application No. 62/978,976, filed on Feb. 20, 2020.

(51) Int. Cl. C12N 9/22; C12Q 1/6806

(52) U.S. Cl. CPC C12N 9/22; C12Y 301/21001; C12Q 1/6806; C07K 2319/30; C07K 2319/31; C07K 2319/50; A61K 38/00

(57) ABSTRACT

The present disclosure provides D1L3 enzymes having complete or partial C-terminal deletions of the basic domain (BD), which have substantially enhanced chromatin-degrading activity. In accordance with aspects of the invention, the D1L3 enzymes described herein are more suitable and/or effective for therapy and/or are more amenable to large-scale manufacturing. In some embodiments, the D1L3 enzymes have benefits for systemic therapy. Such benefits include longer exposure (e.g., slower elimination, longer circulatory half-life), extended duration of pharmacodynamic action, and improved chromatin-degrading activity.

### PRIORITY

This application claims the benefit of, and priority to, U.S. Application Nos. 62/978,976, filed Feb. 20, 2020; 63/040,742, filed Jun. 23, 2020; and 63/064,566, filed Aug. 12, 2020, each of which is hereby incorporated by reference in its entirety.

### BACKGROUND

Inflammation is an essential host response to control invading microbes and heal damaged tissues. Uncontrolled and persistent inflammation causes tissue injury in a plethora of inflammatory disorders. Neutrophils are the predominant leukocytes in acute inflammation. During infections, neutrophils generate neutrophil extracellular traps (NETs), lattices of DNA-filaments decorated with toxic histones and enzymes that immobilize and neutralize bacteria. However, inappropriately released NETs may harm host cells due to their cytotoxic, proinflammatory, and prothrombotic activity.

Two endogenous extracellular DNA-degrading enzymes, DNASE1 (D1) and DNASE1-LIKE 3 (D1L3), limit collateral damage during homeostatic inflammatory responses. Jimenez-Alcázar et al., Host DNases prevent vascular occlusion by neutrophil extracellular traps. Science 358: 1202-1206 (2017). D1 and D1L3 are evolutionarily conserved and found in a variety of species including, humans, primates, and rodents. D1 is predominantly expressed in the gastrointestinal tract and exocrine glands, whereas hematopoietic cells, namely macrophages and dendritic cells produce D1L3. While D1L3 has much higher activity for degrading extracellular chromatin and NETs (as compared to D1, which has little to no chromatin-degrading activity), wild-type D1L3 does not have the physical, enzymatic, or pharmacodynamic properties suitable for therapy.

Therapeutically-effective enzymes with high DNA or chromatin-degrading activity are needed for treating conditions characterized by pathological accumulation of extracellular chromatin, including NETs.

### SUMMARY

D1L3 features a 23-amino acid long C-terminal tail, which contains 9 basic amino acids and is thus known as the Basic Domain (BD). The BD is unique to D1L3 and is not present in D1. The BD contains a nuclear localization signal (NLS) that targets the enzyme to the nucleus during apoptosis. While it has been widely considered that the BD is critical for the biologic activity of D1L3 in the extracellular space, this disclosure surprisingly shows that deletion of the C-terminal tail in fact stimulates chromatinase activity of D1L3.

The BD domain contains three clusters of paired basic amino acids of unknown function, i.e. K291/K292, R297/K298/K299, and K303/R304 in SEQ ID NO: 4. This disclosure shows that heterologous expression of wild-type D1L3 can lead to proteolytic cleavage at these paired basic amino acids and secretion of BD-deleted DNASE1L3 variants. In accordance with aspects of the invention, the D1L3 enzymes described herein are more suitable and/or effective for therapy and/or are more amenable to large-scale manufacturing. The D1L3 enzymes disclosed herein have benefits for systemic therapy. Such benefits include longer exposure (e.g., slower elimination, longer circulatory half-life), extended duration of pharmacodynamic action, and improved chromatin-degrading activity. This disclosure further provides for delivery of D1L3, including D1L3 having BD deletions, using mRNA and gene delivery strategies encoding wild type or engineered version of D1L3.

In various embodiments, the invention provides a DNASE1-LIKE 3 (D1L3) enzyme comprising an amino acid sequence that has at least 70% sequence identity to D1L3 Isoform 1 (SEQ ID NO:4) or D1L3 Isoform 2 (SEQ ID NO:5) lacking the BD (i.e. the amino acids 21 to 282 of SEQ ID NO: 4 or amino acids 21 to 252 of SEQ ID NO: 5), and wherein the D1L3 enzyme has a deletion of one or more amino acids from the BD. Amino acid deletions of the Basic Domain of D1L3 improve its chromatin-degrading activity. Further, increasing deletions of the 23-amino acid BD directly correlate with increasing chromatin-degrading activity, including activity for degrading mono-nucleosomes.

Amino acid deletions of the BD can be anywhere in the BD. For example, deletions can be independently selected from the N-terminal side of the BD, from the C-terminal side of the BD, and internal to the BD. In some embodiments, one or more amino acid deletions are within the NLS.

Additionally, or alternatively, in some embodiments, the BD may comprise amino acid substitutions, which may further impact chromatin-degrading activity. For example, the D1L3 enzyme may have from 1 to 20 amino acid substitutions of BD amino acids (e.g. at least three amino acid substitutions, or at least five amino acid substitutions, or at least 10 amino acid substitutions). Accordingly, in some embodiments, the BD may comprise a combination of amino acid substitution(s) and amino acid deletion(s).

In some embodiments, the D1L3 enzyme is fused to a carrier protein, optionally by means of an amino acid linker. The carrier protein is generally a half-life extending moiety, such as albumin, transferrin, an Fc, or elastin-like protein, XTEN sequence, or a variant thereof.

In some embodiments, the D1L3 enzyme is fused to an albumin amino acid sequence or domain. Albumin can be joined to the D1L3, optionally with an interposed linker, at the N-terminus and/or the C-terminus of the D1L3 enzyme. In some embodiments, the D1L3 enzyme comprises an albumin sequence fused to the N-terminus of the mature D1L3 enzyme with an interposed amino acid linker. The peptide linker may be a flexible linker, a rigid linker, or in some embodiments a physiologically-cleavable linker. An exemplary fusion protein for use in systemic therapy is represented by SEQ ID NO: 47, which includes an N-terminal albumin amino acid sequence, a flexible linker of 31 amino acids, and a mature D1L3 amino acid sequence having a full deletion of the BD. In some embodiments, the D1L3 enzyme is fused to an Fc domain. The Fc domain may be fused to the D1L3 amino acid sequence through a linking sequence. In some embodiments, the D1L3 amino acid sequence is a BDD-D1L3 amino acid sequence, which avoids removal of the Fc domain during expression. In some embodiments, one or more amino acids of the contain mutations to avoid proteolytic digestion during expression or in the systemic circulation. In some embodiments, one or more amino acids of the BD contain mutations to avoid proteolytic digestion during expression or in the systemic circulation. For example, in some embodiments, one or more (or all) of the paired basic amino acids in the BD contain amino acid substitutions to avoid proteolytic digestion during expression or in the systemic circulation.

In still other aspects, the invention provides a DNASE1-LIKE 3 (D1L3) enzyme comprising an amino acid sequence that has at least 70% sequence identity to amino acids 21 to 282 of SEQ ID NO: 4 or amino acids 21 to 252 of SEQ ID NO: 5, and wherein the D1L3 enzyme has a single amino acid truncation of the BD. D1L3 enzymes having a single amino acid truncation from the BD have surprisingly high DNase activity.

The invention in some aspects provides pharmaceutical compositions comprising the D1L3 enzyme described herein, or optionally a polynucleotide encoding the D1L3 enzyme, or a transfection or expression vector comprising the same, or a host cell comprising the polynucleotide or vector, and a pharmaceutically acceptable carrier. The pharmaceutical composition may be formulated for any administration route, including topical, parenteral, or pulmonary administration. In some embodiments, the pharmaceutical composition comprises an isolated cell that is engineered to express and secrete D1L3 in accordance with this disclosure.

In other aspects, the invention provides a method for treating a subject in need of extracellular DNA or chromatin degradation, extracellular trap (ET) degradation and/or neutrophil extracellular trap (NET) degradation. The method comprises administering a therapeutically effective amount of the pharmaceutical composition in accordance with the disclosure. In various embodiments, the present invention provides a method for treating, preventing, or managing diseases or conditions characterized by the presence or accumulation of NETs, including SLE, ARDS (including COVID-19), cancer (including treating or preventing tumor lysis syndrome), and other conditions.

In some embodiments, the present invention pertains to the treatment of diseases or conditions characterized by deficiency of D1L3, or a deficiency of D1. In some cases, the subject has a mutation (i.e., a loss of function mutation) in the Dnase1l3 gene or the Dnase1 gene. In some embodiments, such subjects manifest with an autoimmune disease. In some cases, the subject has an acquired inhibitor of D1 and/or of D1L3. Such subjects can also have an autoimmune or inflammatory disease, such as SLE, scleroderma, systemic sclerosis, or vasculitis.

In some embodiments, the subject has a loss of function mutation in one or both Dnase1l3 genes, and may exhibit symptoms of SLE, vasculitis, or hemolytic anemia, or may be further diagnosed with clinical SLE, urticarial vasculitis, or autoimmune hemolytic anemia. In such embodiments, the subject may receive systemic therapy with D1L3 in accordance with this disclosure, including in some embodiments a BD-deleted D1L3. For example, therapeutically effective amounts of the fusion protein represented by SEQ ID NO:47, or other fusion between albumin or another carrier protein (e.g., an Fc domain) and a BD-deleted D1L3, are administered once or twice weekly, or once or twice monthly. In some embodiments, therapeutically effective amounts of a PEGylated derivative of wild type D1L3 or any variant disclosed herein (without limitation, e.g., a BD-deleted D1L3 variant) are administered. In some embodiments, the PEGylated derivative may have one or more amino acid substitutions in cysteine residues (or PEGylation of Cys residues). In exemplary embodiments, the non-essential cysteines C68 and C194 corresponding to SEQ ID NO:4 is used for site specific PEGylation (PEG, polyethylene glycol).

In other aspects, the invention provides a method for obtaining cell-free DNA (cfDNA) from a subject. As disclosed herein, nucleases such as D1L3 and variants described herein can be administered to subjects to induce substantially higher levels of cfDNA, which can be subsequently isolated for evaluation. In these aspects, the invention provides for methods of cancer screening, including for colorectal cancer, among others. By increasing the levels of cfDNA (including tumor DNA), the invention in some embodiments improves the accuracy (sensitivity or specificity) of cancer screening and evaluation based on cfDNA.

In other aspects, the invention provides a method for treating a subject in need of extracellular chromatin degradation, the method comprising administering a nucleic acid comprising a nucleic acid sequence encoding wild-type DNASE1L3 or a variant thereof. In some embodiments, the nucleic acid is a DNA molecule. In some embodiments, the DNA molecule is a vector or an expression vector, which is optionally, an adeno-associated viral vector (AAV). In other embodiments, the nucleic acid is an mRNA. In some embodiments, the mRNA is a modified mRNA (mmRNA). Without being bound by theory, the nucleic acid is taken up by one or more cells in vivo. In some embodiments, the one or more cells express proteases that cleave one or more positions of the basic domain. In some embodiments, the one or more cells express and secrete a wild-type D1L3 enzyme or a variant thereof. In some embodiments, the one or more cells express and secrete a D1L3 enzyme having a deletion of one or more C-terminal BD amino acids, which leads to enhancement of enzymatic activity.

In some aspects, the invention provides a method for treating a subject in need of extracellular chromatin degradation, the method comprising administering cells that have been manipulated in vitro or ex vivo to express a wild-type DNASE1L3 or a variant thereof. These methods involve (a) transforming a cell in vitro with an exogenous nucleic acid comprising a nucleic acid sequence encoding wild-type DNASE1L3 or a variant thereof, optionally wherein the cell is obtained from the subject; (b) optionally culturing, growing and/or expanding the cell in vitro to generate a progeny of the cell; and (c) administering the cell or the progeny of the cell to the subject. In some embodiments, the cell does not naturally produce and secrete D1L3. In some embodiments, the cell is a T cell, B cell, mesenchymal stem cells, or hematopoietic stem cell. In some embodiments, the cell is a nonadherent cell selected from lymphocyte, monocyte, macrophage, granulocyte, dendritic cell, mesenchymal stem cells, or hematopoietic stem cell. In some embodiments, the cell expresses and secretes a D1L3 enzyme having a deletion of one or more C-terminal BD amino acids, which leads to enhancement of enzymatic activity. In some embodiments, the nucleic acid is a DNA molecule. In some embodiments, the DNA molecule is a vector or an expression vector, which is optionally, an adeno-associated viral vector (AAV). In other embodiments, the nucleic acid is an mRNA. In some embodiments, the mRNA is a modified mRNA (mmRNA).

In some embodiments, the D1L3 variant in accordance with the disclosure has a mutation in the C-terminal basic domain. In some embodiments, the variant has a deletion of at least one amino acid, or at least 3, or at least 5, or at least 8, or at least 9, or at least 13, or at least 14 or at least 15 C-terminal amino acids of the D1L3 basic domain. In some embodiments, the variant has a truncation of at least one amino acid, or at least 3, or at least 5, or at least 8, or at least 9, or at least 13, or at least 14 or at least 15 C-terminal amino acids of the D1L3 basic domain. In some embodiments, the deletion removes one or more of K291, K292, R297, K298, K299, K303, and R304 with respect to SEQ ID NO: 4. In some embodiments, the wild type D1L3 or any variant disclosed herein (without limitation, e.g., a BD-deleted D1L3 variant) may further comprise one or more amino acid substitutions in cysteine residues (or PEGylation of Cys residues). In exemplary embodiments, the non-essential cysteines C68 and C194 corresponding to SEQ ID NO:4 can be used for site specific PEGylation (PEG, polyethylene glycol).

In some embodiments, the nuclease has a fusion or conjugation to a half-life extension moiety, which is optionally an albumin or Fc domain. In some embodiments, the wild-type DNASE1L3 or a variant thereof is capable of secretion from eukaryotic cells.

Other aspects and embodiments of the invention will be apparent from the following examples.

### BRIEF DESCRIPTION OF FIGURES

FIG. 1 shows the enzymatic activity of increasing amounts of DNASE1 (D1) and DNASE1-LIKE 3 (D1L3) as characterized by the degradation of high-molecular weight (HMW)-chromatin from HEK293 cell purified nuclei. D1 has limited activity for degrading chromatin as compared to D1L3.

FIG. 2 shows a schematic representation of a building block substitution technology used for creating chimeric DNase enzymes. Summarily, building block substitution involves protein-protein sequence alignment of a donor DNase (e.g. DNASE1) and a recipient DNase (e.g. DNASE1L3), identification of variable amino acid blocks (building block) between conserved anchors, and substitution of a segment of cDNA encoding a building block from the recipient DNase with a segment of cDNA between the anchors in the donor DNase, thereby creating chimeric DNase enzymes.

FIG. 3 shows 14 of the 63 D1L3-D1 chimeras (#50-#63), SEQ ID NOs: 8-21, respectively, generated using the building block substitution method.

FIG. 4 shows the chromatin-degrading activities of D1L3-D1 chimeras (#50 to #63) shown in FIG. 3. As shown, the D1L3-D1 chimera #63, which harbors a Q282_S305 delinsK mutation (involving deletion of C-terminal basic domain (BD) and substitution of Glutamine 282 of D1L3 with a Lysine) (SEQ ID NO: 21), exhibits hyperactivity for chromatin degradation as compared to wild-type D1L3.

FIG. 5A shows a western blot of culture supernatants using a monoclonal antibody that targets MGDFNAGCSYV (SEQ ID NO:42) (Anti-D1/D1L3), a peptide sequence that is shared by D1 and D1L3. Mobility shift confirms the C-terminal deletion in the Q282_S305 delinsK mutant. FIG. 5B shows the results of a titration experiment performed to compare the chromatin-degrading activities of wild type D1L3 and the BD-deleted D1L3. The results demonstrate that the BD-deleted D1L3 had approximately 5 to 10-fold higher enzymatic activity on HMW-chromatin compared to wild type D1L3. FIG. 5C is a graph showing chromatinase activity of wild type D1L3 and the BD-deleted D1L3 as a function of enzyme concentration.

FIG. 6 shows sequence alignment of C-termini of the S305 del, K303_S305 del, V294_S305 del, K291_S305 del, R285_S305 del, and S283_S305 del deletion mutants (SEQ ID NOs: 22-27, respectively), which lack 1, 3, 12, 15, 21, and 23 C-terminal amino acids, respectively.

FIG. 7A shows the expression systems (CHO or HEK293 Cells) and the methodology (Transient Transfection) used for evaluating the expression of the deletion mutants shown in FIG. 6. FIG. 7B shows a western blot of CHO cell culture supernatants using the anti-D1/D1L3 antibody.

FIG. 8A compares the enzymatic activities of the indicated deletion mutant enzymes with the wild type D1L3 on ultra-large chromatin. FIG. 8B shows a graph illustrating chromatinase activity of D1L3 as a function length of C-terminal deletion.

FIG. 9A shows the ability to degrade ultra-large chromatin into mononucleosomes by the indicated deletion mutant enzymes in comparison with the wild type D1L3. FIG. 9B shows a graph illustrating the degradation of mononucleosomes by the D1L3 mutants as a function length of C-terminal deletion.

FIG. 10 shows the location of NLS2 sequence superimposed with a sequence alignment of C-termini of the S305 del, K303_S305 del, V294_S305 del, K291_S305 del, R285_S305 del, and S283_S305 del deletion mutants, which lack 1, 3, 12, 15, 21, and 23 C-terminal amino acids, respectively.

FIG. 11 shows a western blot of wild type D1L3 and BD-deleted D1L3 expressed in furin-overexpressing CHO cells. CHO cells without overexpression of furin were included as control. Data suggest that cleavage of the C-terminal tail or a portion thereof could act as an activation signal for D1L3.

FIG. 12 compares the enzymatic activities of the indicated deletion mutant enzymes with the wild type D1L3 on DNA.

FIG. 13 shows the use of DNASE1L3-deficient recipient cells (e.g. CHO cell, HEK293 cell, T cell, hepatocyte, Pichia pastoris, Saccharomyces spp.) for expression of nucleic acids encoding for wild-type DNASE1L3 and the secretion of DNASE1L3 enzyme.

FIG. 14A shows the expression vectors containing WT-DNASE1L3 or BDD-DNASE1L3 fused via a linker to Fc fragment, and used for expression in CHO cells.

FIG. 14B shows SDS-PAGE protein gels of Fc fusion proteins purified via protein A/G from supernatants of CHO cells expressing WT-DNASE1L3 or BDD-DNASE1L3 fused via a linker to Fc fragment. Both gels show bands of approximately 65-70 kDa, which reflects the molecular weight of the full-length proteins. An additional lower molecular weight (between 29-44 kDa) is detected, when CHO cells express the WT-DNASE1L3 construct.

FIG. 15 shows the theoretical amino acid sequence of SEQ ID NO: 49 as well as results from N- and C-terminal amino acid sequencing by LC-MS and LC/MS/MS of the upper and lower protein band of WT-DNASE1L3 fused via a linker to Fc fragment shown in FIG. 14B. The N-terminus of the lower band shows one serine residue from the C-terminus of WT-DNASE1L3.

FIG. 16A shows the expression vectors containing WT-DNASE1L3 or WT-DNASE1L3 fused via a linker to albumin used for expression in Pichia pastoris cells.

FIG. 16B shows the results from C-terminal amino acid sequencing by LC-MS and LC/MS/MS of WT-DNASE1L3 and Albumin-(GGS)6-WT-DNASE1L3 fusion protein expressed in Pichia pastoris cells. An intact C-terminus of WT-DNASE1L3 was not detected with either expression construct. C-termini were truncated by 8 to 15 amino acids.

FIG. 17A shows a table of C-terminal amino acid sequences of DNASE1L3 variants that are secreted by cells expressing WT DNASE1L3 cDNA. The C-termini were specifically truncated at the three clusters of paired basic amino acids, resulting in deletions of 1, 8, 9, 13, 14 and 15 amino acids of the BD.

FIG. 17B shows the three clusters of paired basic amino acids within the basic domain of D1L3, i.e. K291/K292, R297/K298/K299, and K303/R304 in SEQ ID NO: 4, that have been identified as sites for post-translational modifications of heterologously expressed wild-type D1L3.

FIG. 18 shows the concentration of cell-free DNA in serum of mice with APAP-induced liver necrosis after injection of vehicle or a recombinant DNASE1L3 variant.

FIG. 19 shows the illustrative work-flow for the enrichment of cell-free DNA in blood samples for diagnostic applications.

### DETAILED DESCRIPTION OF THE INVENTION

The present disclosure is based, in part, on the discovery that D1L3 enzymes having complete or partial C-terminal deletions of the basic domain (BD) have substantially enhanced chromatin-degrading activity. In accordance with aspects of the invention, the D1L3 enzymes described herein are more suitable and/or effective for therapy and/or are more amenable to large-scale manufacturing. In some embodiments, the D1L3 enzymes have benefits for systemic therapy. Such benefits include longer exposure (e.g., slower elimination, longer circulatory half-life), extended duration of pharmacodynamic action, and improved chromatin-degrading activity.

In the description that follows, certain conventions will be followed regarding the usage of terminology. As used herein and in the claims, the singular forms “a,” “an,” and “the” include the singular and the plural reference unless the context clearly indicates otherwise.

The term “chromatinase” refers to a class of deoxyribonuclease enzyme that exhibits more than a negligible ability to cut, cleave or digest chromatin, i.e., DNA associated with one or more histone proteins. Human DNASE1L3 is a chromatinase. DNASE1L3 variants disclosed herein are chromatinases. Not all DNASE enzymes are chromatinases. For example, human DNASE1 (D1) has essentially no ability to specifically cut, cleave, or digest chromatin and is not a chromatinase.

As used herein, unless stated to the contrary, the term “D1L3” when referring to the wild-type sequence, includes either D1L3 Isoform 1 (SEQ ID NO:4) or D1L3 Isoform 2 (SEQ ID NO:5).

When referring to sequence identity with wild-type DNase enzymes, and unless stated otherwise, sequences refer to mature enzymes lacking the signal peptide. Further, unless stated otherwise, amino acid positions are numbered with respect to the full-translated DNase sequence, including signal peptide, for clarity. Accordingly, for example, reference to sequence identity to the enzyme of SEQ ID NO: 4 (human D1L3, Isoform 1) refers to a percent identity with the mature enzyme having M21 at the N-terminus.

As used herein with reference to a drug, “half-life” refers to the elimination half-life of the concentration of the drug in an animal, as measured in a matrix of interest, e.g., serum or plasma. The skilled person will understand that not all drugs exhibit first-order kinetics or do so during all phases of elimination. In such cases, the skilled person will understand that the terms “half-life extension” or “extended half-life” are expressions that refer to a slower rate of elimination.

As used herein, “neutrophil extracellular trap” and the acronym “NET” refer to a network of extracellular fibers comprising nuclear contents, e.g., DNA bound to histone proteins that are released from an immune cell, typically a neutrophil, in a programmed fashion.

Unless otherwise specified, a “nucleotide sequence” or “nucleic acid” encoding an amino acid sequence includes all degenerate versions that encode the same amino acid sequence. The phrase nucleotide sequence that encodes a protein or an RNA may also include introns to the extent that the nucleotide sequence encoding the protein may in some version contain one or more introns.

The terms “about” and “approximately” include an amount that is +10% of an associated numerical value.

D1L3 features a 23-amino acid long C-terminal tail, which contains 9 basic amino acids and is thus known as Basic Domain (BD). The BD is unique to D1L3 and is not present in D1. The BD contains a nuclear localization signal (NLS) that is believed to target the enzyme to the nucleus during apoptosis. While it has been widely considered that the BD is also critical for the biologic activity of D1L3 in the extracellular space, this disclosure surprisingly shows that deletion of the C-terminal tail in fact stimulates chromatinase activity of D1L3.

The BD domain contains three clusters of paired basic amino acids of unknown function, i.e. K291/K292, R297/K298/K299, and K303/R304 in SEQ ID NO: 4. This disclosure shows that heterologous expression of wild-type D1L3 can lead to proteolytic cleavage at these paired basic amino acids and secretion of DNASE1L3 variants having BD deletions.

In various embodiments, the invention provides a DNASE1-LIKE 3 (D1L3) enzyme comprising an amino acid sequence that has at least 70% sequence identity to D1L3 Isoform 1 (SEQ ID NO:4) or D1L3 Isoform 2 (SEQ ID NO:5) lacking the BD (i.e., at least 70% sequence identity with amino acids 21 to 282 of SEQ ID NO: 4, or amino acids 21 to 252 of SEQ ID NO:5), and wherein the D1L3 enzyme has a deletion of one or more amino acids from the BD. Amino acid deletions of the Basic Domain of D1L3 improve its chromatin-degrading activity. Further, increasing deletions of the 23-amino acid BD directly correlate with increasing chromatin-degrading activity. In some embodiments, the D1L3 enzyme variant having a deletion of at least one amino acid, or at least 3, or at least 5, or at least 8, or at least 9, or at least 13, or at least 14 or at least 15 C-terminal amino acids of the D1L3 basic domain have increased ability to degrade mono-nucleosomes.

In various embodiments, the amino acid deletions from the BD are at the C-terminus of the BD. For example, the D1L3 enzyme may have a deletion of at least the five C-terminal amino acids of the BD. In some embodiments, the D1L3 enzyme has a deletion of at least the eight C-terminal amino acids of the BD. In some embodiments, the D1L3 enzyme has a truncation of at least the eight C-terminal amino acids of the BD. In some embodiments, the D1L3 enzyme has a deletion of at least the ten C-terminal amino acids of the BD. In some embodiments, the D1L3 enzyme has a truncation of at least the ten C-terminal amino acids of the BD. In some embodiments, the D1L3 enzyme has a deletion of at least the twelve C-terminal amino acids of the BD. In some embodiments, the D1L3 enzyme has a truncation of at least the twelve C-terminal amino acids of the BD. In some embodiments, the D1L3 enzyme has a deletion of at least the fifteen C-terminal amino acids of the BD. In some embodiments, the D1L3 enzyme has a truncation of at least the fifteen C-terminal amino acids of the BD. In some embodiments, the D1L3 enzyme has a deletion of at least the eighteen C-terminal amino acids of the BD. In some embodiments, the D1L3 enzyme has a truncation of at least the eighteen C-terminal amino acids of the BD. In some embodiments, the D1L3 enzyme has a deletion of at least the twenty-one C-terminal amino acids of the BD. In some embodiments, the D1L3 enzyme has a truncation of at least the twenty C-terminal amino acids of the BD. In some embodiments, the D1L3 enzyme has a deletion of at least the twenty-three C-terminal amino acids of the BD. In some embodiments, the D1L3 enzyme has a truncation of at least the twenty-three C-terminal amino acids of the BD. In some embodiments, the D1L3 enzyme has a deletion of at least the C-terminal serine of the BD. In some embodiments, the D1L3 enzyme has a truncation of C-terminal serine of the BD.

Alternatively, deletions of the BD (e.g., from three to 23 amino acids) can be anywhere in the BD, and not necessarily from the C-terminus of the BD. For example, in various embodiments, the D1L3 enzyme has 1, 2, 3, 4, 5, 6, 7, 8, 9, 10, 11, 12, 13, 14, 15, 16, 17, 18, 19, 20, 21, 22, or 23 amino acids deleted from the BD. In some embodiments, the D1L3 enzyme has a deletion of at least 1, or at least 3, or at least 5, or at least 8, or at least 12, or at least 15, or at least 18, or at least 21 amino acids from the BD. In some embodiments, the D1L3 enzyme has a truncation of at least 1, or at least 3, or at least 5, or at least 8, or at least 12, or at least 15, or at least 18, or at least 21 amino acids from the BD. These deletions can be independently selected from the N-terminal side of the BD, from the C-terminal side of the BD, and internal to the BD. In some embodiments, one or more amino acid deletions are within the NLS. In some embodiments, the deleted amino acid is the C-terminal serine of the BD. In some embodiments, the deletion is sufficient to remove all paired basic amino acids in the BD from the enzyme.

In addition to deletions of one or more amino acids, the BD may further comprise amino acid substitutions, which may further impact chromatin-degrading activity. For example, the D1L3 enzyme may have from 1 to 20 amino acid substitutions of BD amino acids, in addition to a deletion of at least three amino acids. In some embodiments, the BD contains a substitution of at least three amino acids, or at least five amino acids, or at least 10 amino acids. In some embodiments, at least two amino acid substitutions are in the NLS of the BD. In some embodiments, one or more paired basic amino acids in the BD are substituted to prevent cleavage. In such embodiments, a more homogeneous enzyme may be expressed and secreted, e.g., for recombinant enzyme production.

In some embodiments, the D1L3 enzyme has a deletion of one or more additional amino acids from the C-terminus, in addition to a deletion of the BD. For example, the D1L3 enzyme may have a deletion of an additional one to fifty amino acids, or from one to twenty amino acids, or from one to ten amino acids, or from one to five amino acids from the C-terminal amino acids of SEQ ID NO:4 or SEQ ID NO:5, in addition to the deletion of the BD.

In some embodiments, after partial or complete deletion of the BD as described, from 1 to 10 amino acids, or from 1 to 5 amino acids may be added to the C-terminus that do not impact chromatin-degrading activity.

In some embodiments, the wild type D1L3 or any variant disclosed herein (without limitation, e.g., a BD-deleted D1L3 variant) may further comprise one or more amino acid substitutions in cysteine residues (or PEGylation of Cys residues). In exemplary embodiments, the non-essential cysteines C68 and C194 corresponding to SEQ ID NO:4 can be used for site specific PEGylation (PEG, polyethylene glycol). In some embodiments, the recombinant D1L3 protein variant comprises one or more polyethylene glycol (PEG) moieties, which may be conjugated at one or more positions or the C-terminus. In some embodiments, the native amino acid at that position is substituted with an amino acid having a side chain suitable for crosslinking with hydrophilic moieties, to facilitate linkage of the hydrophilic moiety to the peptide. In other embodiments, an amino acid modified to comprise a hydrophilic group is added to the peptide at the C-terminus. The PEG chain(s) may have a molecular weight in the range of about 500 to about 40,000 Daltons. In some embodiments, the PEG chain(s) have a molecular weight in the range of about 500 to about 5,000 Daltons. In some embodiments, the invention provides a D1L3 enzyme having a polyethylene glycol (PEG) moiety conjugated at the position corresponding to Cys 68 corresponding to SEQ ID NO: 4, which is believed to be an unpaired cysteine. In some embodiments, the D1L3 variant has a PEG conjugation to the amino acid corresponding to C194 corresponding to SEQ ID NO: 4. In these embodiments, the PEG moiety will provide a half-life extension property, while avoiding disulfide scrambling and/or protein misfolding. In some embodiments, the PEG moiety is conjugated through maleimide chemistry, which can be conducted under mild conditions. Other conjugation chemistries are known and may be used, such as vinyl sulfone, dithyopyridine, and iodoacetamide activation chemistries. The PEG moiety can be linear or branched, and can be generally in the range of 10 kDa to 40 kDa, or in the range of 20 to 30 kDa. In some embodiments, the PEG chain(s) have a molecular weight of about 10,000 to about 20,000 Daltons. PEGylation is disclosed in WO 2019/036719 and WO 2020/076817, both of which are hereby incorporated by reference in its entirety.

In various embodiments, the D1L3 enzyme comprises an amino acid sequence having at least 80% sequence identity to amino acids 21 to 282 of SEQ ID NO: 4 or amino acids 21 to 252 of SEQ ID NO: 5. In some embodiments, the D1L3 enzyme comprises an amino acid sequence having at least 85% sequence identity to amino acids 21 to 282 of SEQ ID NO: 4 or amino acids 21 to 252 of SEQ ID NO: 5. In some embodiments, the D1L3 enzyme comprises an amino acid sequence having at least 90% sequence identity to amino acids 21 to 282 of SEQ ID NO: 4 or amino acids 21 to 252 of SEQ ID NO: 5. In such embodiments, the amino acid sequence may have at least 95% sequence identity to amino acids 21 to 282 of SEQ ID NO: 4 or amino acids 21 to 252 of SEQ ID NO: 5, or at least 97% sequence identity to amino acids 21 to 282 of SEQ ID NO: 4 or amino acids 21 to 252 of SEQ ID NO: 5. In some embodiments, the D1L3 enzyme comprises an amino acid sequence having 100% sequence identity with the enzyme of SEQ ID NO:4 or SEQ ID NO:5 lacking the BD.

In still other aspects, the invention provides a DNASE1-LIKE 3 (D1L3) enzyme comprising an amino acid sequence that has at least 70% sequence identity to D1L3 Isoform 1 (SEQ ID NO:4) or D1L3 Isoform 2 (SEQ ID NO:5) (i.e. the amino acids 21 to 282 of SEQ ID NO: 4 or amino acids 21 to 252 of SEQ ID NO: 5), and wherein the D1L3 enzyme has a single amino acid truncation of the BD. D1L3 enzymes having a single amino acid truncation from the BD have surprisingly high DNase activity.

In some embodiments, the D1L3 enzyme comprises additional modifications outside the BD, and which can provide additional advantages, including advantages in stability and compatibility with expression systems. Such modifications are disclosed in US 2020/0024585, U.S. 2020/0115690, or PCT/US2020/016490, each of which are hereby incorporated by reference in its entirety.

In some embodiments, the D1L3 enzyme comprises at least one building block substitution from D1 (SEQ ID NO:1), DNASE-1-LIKE 1 (D1L1) (SEQ ID NO:2), or DNASE-1-LIKE 2 (SEQ ID NO:3). These building block substitutions are disclosed in PCT/US2020/016490, which is hereby incorporated by reference in its entirety.

In some embodiments, the D1 L3 sequence or domain contains a building block substitution from D1 defined by amino acid sequences, which can be selected from: M1_A20 delinsMRGMKLLGALLALAALLQGAVS, M21_S25 delinsLKIAA, V28_S30 delinsIQT, E33_S34 delinsET, Q36_I45 delinsMSNATLVSYI, K47_K50 delinsQILS, C52Y, I54_M58 delinsIALVQ, I60_K61 delinsVR, S63_I70 delinsSHLTAVGK, M72_K74 delinsLDN, R77_T84 delinsQDAPDT, N86H, V88_I89 delinsVV, S91_R92 delinsEP, N96_T97 delinsNS, Q101R, A103L, L105V, K107_L110 delinsRPDQ, V113_S116 delinsAVDS, H118Y, H120D, Y122_A127 delinsGCEPCGN, V129T, S131N, 135F_136 VdelinsAI, W138R, Q140_H143 delinsFSRF, A145_D148 delinsAVKD, V150A, I152A, T156_T157 delinsAA, E159_S161 delinsGDA, K163A, E167A, V169_E170 delinsYD, T173L, K176_R178 delinsQEK, K180_A181 delinsGL, N183_F186 delinsDVML, P198_A201 delinsRPSQ, K203_N204 delinsSS, R208W, D210S, R212T, V214Q, G218P, Q220_E221 delinsSA, V225_S228 delinsATP, N230H, L238_R239 delinsVA, Q241_S246 delinsMLLRGA, K250D, N252_V254 delinsALP, D256N, K259_A260 delinsAA, K262G, T264_E267 delinsSDQL, L269_V271 delinsQAI, F275Y, F279_K280 delinsVM, and Q282_S305 delinsK.

In some embodiments, the D1L3 enzyme is fused to a carrier protein, optionally by means of an amino acid linker. The carrier protein is generally a half-life extending moiety, such as albumin, transferrin, an Fc domain, XTEN (see U.S. Pat. No. 8,492,530 which is hereby incorporated by reference in its entirety), or elastin-like protein, or a variant thereof. See, e.g., U.S. Pat. No. 9,458,218, which is hereby incorporated by reference in its entirety.

In some embodiments, the D1L3 enzyme is fused to an albumin amino acid sequence or domain, i.e., a human albumin or a fragment or variant thereof. See, for example, WO 2015/066550 and U.S. Pat. No. 9,221,896, which are hereby incorporated by reference in their entirety. Albumin can be joined to the D1L3, optionally with an interposed linker, at the N-terminus and/or the C-terminus of the D1L3 enzyme. An exemplary albumin amino acid sequence is provided by SEQ ID NO: 39. In some embodiments, the D1L3 enzyme comprises an albumin sequence fused to the N-terminus and/or C-terminus of the mature D1L3 enzyme with an interposed amino acid linker. Linker constructs are described in detail herein.

In some embodiments, the albumin amino acid sequence or domain of the fusion protein has at least about 75%, or at least about 80%, or at least about 85%, at least 90%, at least 95%, at least 97%, at least 98%, or at least 99% sequence identity to the reference albumin sequence defined by SEQ ID NO: 39. In some embodiments, the albumin amino acid sequence or domain comprises or consists of the reference albumin sequence defined by SEQ ID NO: 39. In various embodiments, the albumin amino acid sequence binds to the neonatal Fc receptor (FcRn), e.g., human FcRn. The albumin amino acid sequence may be a variant of wild-type HSA (e.g., as represented by SEQ ID NO: 39). In various embodiments, albumin variants may have from one to twenty, or from one to ten amino acid modifications independently selected from deletions, substitutions, and insertions with respect to SEQ ID NO: 39. In some embodiments, the albumin amino acid sequence is any mammalian albumin amino acid sequence. Various modification to the albumin sequence that enhance its ability to serve as a circulation half-life extending carrier are known, and such modifications can be employed with the present invention. Exemplary modifications to the albumin amino acid sequence are described in U.S. Pat. Nos. 8,748,380, 10,233,228, 10,208,102, and 10,501,524, which are each hereby incorporated by reference in their entireties. Exemplary modifications include one or more (or all) of E505Q, T527M, and K573P as described therein.

In some embodiments, the albumin amino acid sequence or domain is a fragment of full-length albumin, as represented by SEQ ID NO: 39. The term “fragment,” when used in the context of albumin, refers to any fragment of full-length albumin or a variant thereof (as described above) that extends the half-life of a D1L3 enzyme to which it is fused or conjugated, relative to the corresponding non-fused D1L3. In some embodiments, a fragment of an albumin can refer to an amino acid sequence comprising a fusion of multiple domains of albumin (see, e.g., WO2011/124718), such as domains I and III, and domains II and III. Generally, a fragment of albumin has at least about 100 amino acids or at least about 200 or at least about 300 amino acids of the full-length sequence. In various embodiments, the albumin fragment maintains the ability to bind human FcRn.

In some embodiments, the D1L3 enzyme is fused at the N-terminus to an albumin amino acid sequence, through a peptide linker. The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length. In some embodiments, the linker is from about 10 to about 35 amino acids in length, or from about 15 to about 35 amino acids. In some embodiments, the linker is a flexible linker of from 20 to 40 amino acids. Flexible linkers can comprise predominately (or consist of) Gly and Ser amino acid residues.

In some embodiments, a DNA sequence encoding albumin may be cloned from a variety of genomic or cDNA libraries known in the art, and used to construct polynucleotides encoding fusion proteins of the D1L3 enzyme or any variant of the D1L3 enzyme disclosed herein. In some embodiments, the gene encoding fusion proteins of the D1L3 enzyme or any variant of the D1L3 enzyme disclosed herein and an albumin may be cloned into a vector or expression vector. In some embodiments, the vector or expression vector comprising a gene encoding fusion proteins of the D1L3 enzyme or any variant of the D1L3 enzyme disclosed herein and an albumin may be used for therapy. In some embodiments, cells may be transformed in vitro or ex vivo with the vector or expression vector and used for therapy. For example, the transformed cells may be grown and/or expanded in vitro; and the in vitro grown/expanded cells may be used in therapy (administered to a patient). In some embodiments, the cell is a cell that does not naturally produce D1L3. Exemplary cells include T cells, B cells, and hematopoietic stem cells. In some embodiments, the D1L3 fusion is expressed in a macrophage or dendritic cell (e.g., a cell that naturally produces some amount of D1L3). In these embodiments, the amount of D1L3 present in circulation is substantially enhanced by complementation of the gene for cell therapy.

In some embodiments, a modified mRNA (mmRNA) encoding fusion proteins of the D1L3 enzyme or any variant of the D1L3 enzyme disclosed herein and an albumin may be used in therapy. In some embodiments, the modified mRNA (mmRNA) encodes a wild type D1L3 enzyme (i.e. without any of the mutations disclosed herein and without a fusion partner). In some embodiments, the modified mRNA (mmRNA) encodes any variant of the D1L3 enzyme disclosed herein (i.e. including truncations, deletions, or substitutions disclosed herein, with or without a fusion partner). In some embodiments, the modified mRNA (mmRNA) encodes a fusion protein of wild type D1L3 enzyme with a fusion partner. In some embodiments, the modified mRNA (mmRNA) encodes a fusion protein of any of the D1L3 enzyme variants disclosed herein (i.e. with any of the mutations including truncations, deletions, or substitutions disclosed herein) and a fusion partner (without limitation, e.g., albumin and Fc). In some embodiments, cells may be transformed in vitro or ex vivo with a mmRNA encoding the D1L3 enzyme (or any variant of the D1L3 enzyme disclosed herein), and used for therapy. That is, cells may be grown and/or expanded in vitro; and transformed either before or after expansion with mRNA encoding the D1L3 enzyme. The in vitro expanded and transformed cells may be used in therapy.

In some embodiments, the D1L3 enzyme is fused to an Fc domain. See, for example, WO 2005047334A1, WO 2004074455A2, U.S. 20070269449, which are hereby incorporated by reference in their entirety. In some embodiments, the human Fc domain is selected from IgG1, IgG2, IgG3, and IgG4. In some embodiments, the human Fc domain is a human IgG Fc domain. Fc domain can be joined to the D1L3, optionally with an interposed linker, at the N-terminus and/or the C-terminus of the D1L3 enzyme. In some embodiments, the D1L3 enzyme comprises an Fc domain sequence fused to the N-terminus of the mature D1L3 enzyme with an interposed amino acid linker. The peptide linker may be a flexible linker, a rigid linker, or in some embodiments a physiologically-cleavable linker as described herein. In some embodiments, the D1L3 enzyme comprises an Fe domain sequence fused to the C-terminus of the D1L3 enzyme, optionally through a linker (e.g., a flexible linker). In some embodiments, the D1L3 enzyme (with Fc fused at the C-terminus) has a mutation in one or more amino acids of the BD that reduces or eliminates proteolytic cleavage (without limitation, e.g., substitutions of paired basic amino acids). In some embodiments, the D1L3 enzyme (with Fc fused at the C-terminus) has a deletion of the BD to remove the paired basic amino acids in the BD that are otherwise substrates for proteolytic cleavage.

In some embodiments, an Fc domain may be joined to the D1L3, optionally with an interposed linker, at the N-terminus and/or the C-terminus of the D1L3 enzyme. In some embodiments, the Fc domain has at least two heavy chain constant region domains (CH2 and CH3) and a hinge region. In some embodiments, the Fc domain is selected from IgG1, IgG2, IgG3, and IgG4. In some embodiments, the Fc domains is a human Fc domain. In some embodiments, the Fc domains is a human IgG Fc domain. Fc domain can be joined to the D1L3, optionally with an interposed linker, at the N-terminus and/or the C-terminus of the D1L3 enzyme. In some embodiments, the D1L3 enzyme comprises an Fc domain sequence fused to the N-terminus of the mature D1L3 enzyme with an interposed amino acid linker. The peptide linker may be a flexible linker, a rigid linker, or in some embodiments a physiologically-cleavable linker. In some embodiments, altered forms of Fc domains having improved serum half-life, altered effector functions, altered spatial orientation, and the like are used.

In some embodiments, the D1L3 enzyme is fused to an albumin. Albumin can be joined to the D1L3, optionally with an interposed linker, at the N-terminus and/or the C-terminus of the D1L3 enzyme. In some embodiments, the albumin is a human serum albumin. In some embodiments, the D1L3 enzyme comprises an albumin sequence fused to the C-terminus of the mature D1L3 enzyme with an interposed amino acid linker. The peptide linker may be a flexible linker, a rigid linker, or in some embodiments a physiologically-cleavable linker as described herein. In some embodiments, the D1L3 enzyme (with albumin fused at the C-terminus) has a mutation in amino acids of the BD that enable proteolytic cleavage. In some embodiments, the D1L3 enzyme (with Fc fused at the C-terminus) has a deletion of the BD to remove the paired basic amino acids in the BD that are otherwise substrates for proteolytic cleavage.

In some embodiments, a DNA sequence encoding Fc domain may be cloned from a variety of genomic or cDNA libraries known in the art in frame with a gene encoding the D1L3 enzyme or any variant of the D1L3 enzyme disclosed herein. In some embodiments, the gene encoding the fusion protein of the D1L3 enzyme or any variant of the D1L3 enzyme disclosed herein and an Fc domain may be cloned in a vector or expression vector. In some embodiments, the vector or expression vector comprising a gene encoding the fusion protein of the D1L3 enzyme or any variant of the D1L3 enzyme disclosed herein and an Fc domain may be used for therapy. In some embodiments, cells may be transformed in vitro or ex vivo with the vector or expression vector comprising a gene encoding fusion protein of the D1L3 enzyme or any variant of the D1L3 enzyme disclosed herein and an Fc domain, and the cells may be used for therapy. That is, the transformed cells may be grown and/or expanded in vitro; and the in vitro grown/expanded cells may be used in therapy. In some embodiments, the cell is a cell that does not naturally produce D1L3. Exemplary cells include T cells, B cells, and hematopoietic stem cells. In some embodiments, the D1L3 fusion is expressed in a macrophage or dendritic cell (e.g., a cell that naturally produces some amount of D1L3). In these embodiments, the amount of D1L3 present in circulation is substantially enhanced by complementation of the gene for cell therapy.

In some embodiments, a modified mRNA (mmRNA) encoding the fusion protein of the D1L3 enzyme or any variant of the D1L3 enzyme disclosed herein and an Fc domain may be used in therapy. In some embodiments, cells may be transformed in vitro or ex vivo with an mmRNA encoding fusion proteins of the D1L3 enzyme (or any variant of the D1L3 enzyme disclosed herein) and an Fc domain, and used for therapy. That is, cells may be grown and/or expanded in vitro; and transformed either before or after expansion with mRNA (e.g., mmRNA) encoding the D1L3 fusion protein. The in vitro expanded and transformed cells may be used in therapy.

Flexible linkers are predominately or entirely composed of small, non-polar or polar residues such as Gly, Ser and Thr. An exemplary flexible linker comprises (Gly_{y}Ser)ₙS_{z} linkers, where y is from 1 to 10 (e.g., from 1 to 5), n is from 1 to about 10, and z is 0 or 1. In some embodiments, n is from 3 to about 8, or from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 3 to 6). Generally, linkers can be predominately composed of amino acids selected from Gly, Ser, Thr, Ala, and Pro. Exemplary linker sequences contain at least 10 amino acids, and may be in the range of 10 to about 50 amino acids, or about 15 to about 40 amino acids, or about 15 to about 35 amino acids. Exemplary linker designs are provided as SEQ ID NOS: 31 to 38.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise or consist of S(GGS)₄GSS (SEQ ID NO: 36), S(GGS)₉GSS (SEQ ID NO: 37), (GGS)₉GSS (SEQ ID NO: 38). In some embodiments, the linker has at least 10 amino acids, or at least 15 amino acids, or at least 20 amino acids, or at least 25 amino acids, or at least 30 amino acids. For example, the linker may have a length of from 15 to 40 amino acids. In various embodiments, longer linkers of at least 15 amino acids can provide improvements in yield upon expression in Pichia pastoris. See PCT/US2019/055178, which is hereby incorporated by reference in its entirety.

An exemplary fusion protein for use in systemic therapy is shown as SEQ ID NO: 47, which includes an N-terminal albumin amino acid sequence, a flexible linker of 31 amino acids, and a mature D1L3 amino acid sequence having a full deletion of the BD.

In other embodiments, the linker is a physiologically-cleavable linker, such as a protease-cleavable linker. For example, the protease may be a coagulation pathway protease, such as activated Factor XII. In some embodiments, the linker comprises the amino acid sequence of Factor XI (SEQ ID NO: 40) and/or prekallikrein (SEQ ID NO: 41) or a physiologically cleavable fragment thereof. In selected embodiments, the linker amino acid sequence from Factor XI contains all or parts of SEQ ID NO: 40 (e.g., parts of SEQ ID NO: 40, including modifications of SEQ ID NO: 40 that allow for cleavage by Factor XIIa). In some embodiments, the linker amino acid sequence from prekallikrein contains all or parts of SEQ ID NO: 41 (e.g., parts of SEQ ID NO: 41, including modifications of SEQ ID NO: 41 that allow for cleavage by Factor XIIa). In other embodiments, the linker includes a peptide sequence that is targeted for cleavage by a neutrophil specific protease, such as neutrophil elastase, cathepsin G, and proteinase 3.

The chromatin- and/or NET-degrading activity of a D1L3 enzyme variant, e.g., comprising a deletion of one or more amino acids of the BD, can be measured in vitro, for example by incubation of the enzyme with chromatin or NETs. Chromatin or NETs can be obtained in some embodiments from purified nuclei or ex vivo blood or neutrophils induced to form NETs. Alternatively, the chromatin- and/or NET-degrading activity of an enzyme can be measured in vivo, for example by administering the enzyme to a subject, wherein the subject produces or is induced to produce extracellular DNA, chromatin or NETs, and measuring the effect of the enzyme on concentrations of DNA, chromatin, or NET levels in a matrix, e.g. serum, preferably with a parallel negative control, or by temporally comparing the concentrations before and after administration of the enzyme.

In some embodiments, the fusion protein is synthesized with an N-terminal signal peptide. The signal peptide may be removed during secretion from the host cell. With respect to expression in Pichia pastoris, the alpha-mating factor (αMF) pre-pro secretion leader from Saccharomyces cerevisiae (SEQ ID NO: 28) may be used for expression. In other embodiments, the signal peptide and propeptide of HSA, which consists of a signal sequence of 24 amino acids (MKWVTFISLLFLFSSAYSRGVFRR; SEQ ID NO: 29) may be used. In some embodiments, the human DNASE1L3 Signal Peptide (Q13609) (SEQ ID NO: 30) is used for expression. These elements are cleaved during expression, and are not present in the D1L3 enzyme product.

The invention in some aspects provides pharmaceutical compositions comprising the D1L3 enzyme described herein, or optionally a polynucleotide encoding the D1L3 enzyme, or a transfection or expression vector comprising the same, or a cell comprising the polynucleotide or vector, and a pharmaceutically acceptable carrier.

In some embodiments, delivery of polynucleotides is used for therapy. Encoding polynucleotides can be delivered as mRNA or as DNA constructs using known procedures, e.g., electroporation or cell squeezing, and/or vectors (including viral vectors). mRNA polynucleotides can include known modifications (mmRNA) to avoid activation of the innate immune system. See WO 2014/028429, which is hereby incorporated by reference in its entirety. In some embodiments, the polynucleotide is delivered to the body of a subject. In some embodiments, the polynucleotides is delivered into a cell in vitro, and the cell is delivered to the body of a subject. The cell can be, for example, a white blood cell (e.g., a T cell, B cell, or macrophage), an endothelial cell, an epithelial cell, a hepatocyte, a fibroblast, or a stem cell (e.g., a hematopoietic stem cell).

In some embodiments, the polynucleotide used for therapy is a modified mRNA (mmRNA). In some embodiments, the mmRNA is administered to a subject in need of treatment. In some embodiments, the cells are transformed with a modified mRNA (mmRNA) in vitro or ex vivo, expanded before or after transfection, and used for therapy (cell therapy). In some embodiments, the mmRNAs may be uniformly modified along the entire length of the molecule. In alternative embodiments, the mmRNAs may not be uniformly modified along the entire length of the molecule. Different nucleotide modifications and/or backbone structures may exist at various positions in the nucleic acid. In some embodiments, the nucleotide analogs or other modification(s) may be located at any position(s) of a nucleic acid such that the function of the nucleic acid is not substantially decreased. In some embodiments, the mmRNAs may comprise a 5′ or 3′ terminal modification.

In some embodiments, the mmRNA may contain at least about 5% modified nucleotides, or at least about 10% modified nucleotides, or at least about 20% modified nucleotides, or at least about 50% modified nucleotides, at least about 80% modified nucleotides. In some embodiments, the mmRNA may contain less than about 10% modified nucleotides, or less than about 20% modified nucleotides, or less than about 50% modified nucleotides.

In some embodiments, the mmRNA may include a polynucleotide modification such as, but not limited to, a nucleoside modification. The nucleoside modification may include, but is not limited to, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof. Suitable modifications are disclosed in U.S. 20190060458, the contents of which are hereby incorporated by reference in its entirety.

In some embodiments, the polynucleotide used for therapy is a DNA molecule encoding a wild type D1L3 enzyme or any variant of D1L3 disclosed herein (i.e., gene therapy). In some embodiments, the cells are transformed with a DNA molecule encoding a wild type D1L3 enzyme or any variant of D1L3 disclosed herein in vitro or ex vivo, expanded, and used for therapy (i.e., cell therapy). In some embodiments, the DNA molecule is a vector. A vector generally comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In some embodiments, the vector is a viral vector. Exemplary vectors include autonomously replicating plasmids or a virus (e.g. AAV vectors). The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In some embodiments, the polynucleotide or cell therapy may employ expression vectors, which comprise the nucleic acid encoding the chromatinase (e.g., D1L3) operably linked to an expression control region that is functional in the host cell. The expression control region is capable of driving expression of the operably linked encoding nucleic acid such that the chromatinase is produced in a human cell transformed with the expression vector. Expression control regions are regulatory polynucleotides (sometimes referred to herein as elements), such as promoters and enhancers, that influence expression of an operably linked nucleic acid. An expression control region of an expression vector is capable of expressing operably linked encoding nucleic acid in a human cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. In various embodiments, the chromatinase expression is inducible or repressible. Typically, the amount of increase or decrease conferred by such elements is proportional to the amount of signal present; the greater the amount of signal, the greater the increase or decrease in expression.

In some embodiments, the viral vector is an adeno-associated viral vector (AAV). In some embodiments, suitable AAV-based vectors in the current disclosure have very limited capacity to induce immune responses in humans. The AAV genome is typically built of single-stranded deoxyribonucleic acid (ssDNA), either positive- or negative-sensed, which is about 4.7 kilobases long. The AAV genome comprises inverted terminal repeats (ITRs) at both ends of the DNA strand, and two open reading frames (ORFs): rep and cap. Development of AAVs as gene therapy vectors has eliminated the integrative capacity of the vector by removal of the rep and cap from the DNA of the vector. In some embodiments, a gene encoding a wild type D1L3 enzyme or any variant of D1L3 disclosed herein, which is operably linked to a promoter, may be inserted between the inverted terminal repeats (ITR). In some embodiments, the AAV vector comprising a wild type D1L3 enzyme or any variant D1L3 disclosed herein may form a concatemer in the nucleus after the single-stranded vector DNA is converted by host cell DNA polymerase complexes into double-stranded DNA. In some embodiments, the AAV vector comprising a wild type D1L3 enzyme or any variant D1L3 disclosed herein may thus form episomal concatemers in the host cell nucleus. In some embodiments, the concatemers may remain intact for the life of the non-dividing host cell. In some embodiments, the concatemers may be lost through cell division dividing cells.

In an illustrative embodiment, the AAV serotype 8 (AAV2/8) vector is used. In some embodiments, the recombinant AAV serotypes used for delivery of the polynucleotide are replication-defective, generally do not insert into the host genome and show a lack of pathogenicity and immune response in human subjects. Any AAV vector may be used, including, but not limited to, AAV1, AAV2, AAV3, AAV4, AAV5, AAV6, AAV7, AAV8 and combinations thereof. In some instances, the AAV comprises LTRs that are of a heterologous serotype in comparison with the capsid serotype (e.g., AAV2 ITRs with AAV5, AAV6, or AAV8 capsids).

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription-initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J Mol. Biol. 335:667-678, 2004), sleeping beauty, transposases of the mariner family, and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences including CRISPR/CAS9, zinc finger, TALEN, and meganuclease gene-editing technologies.

Thus, in some embodiments, the invention provides mammalian host cells (e.g., human host cells), as well as methods of making and using the same. The host cells comprise a heterologous polynucleotide encoding a chromatinase enzyme (as described). The host cells delivered to a subject express and secrete the encoded chromatinase enzyme. In these aspects, challenges in manufacturing chromatinases such as D1L3 at large scale are avoided. Further, by expressing and delivering D1L3 through heterologous expression in a white blood cell such as a T cell, B cell or macrophage, or a fibroblast, D1L3 therapy can be localized in part to areas of inflammation or tissue destruction or cell apoptosis or wound healing. Further, since WT D1L3 has a circulation half-life of less than about 30 minutes, the cell therapy described herein provides for a sustained therapy, with as few as one, two, three, or four treatments in some embodiments. In some embodiments, the therapy is provided to a subject for treatment of cancer (e.g., leukemia) or viral infection, including infection of the lower respiratory tract. In some embodiments, the host cells are created from cells of the subject to be treated or an HLA-matched donor. In some embodiments, the cells are HLA null, or are created from HLA-matched source cells.

In some embodiments, the cell therapy may employ cells harboring a chimeric antigen receptor protein (CAR), which include, without limitation, e.g. CAR T cells or CAR NK cells. Any of the variants of CAR T cells or CAR NK cells may be employed. Hartmann et al., Clinical development of CAR T cells—challenges and opportunities in translating innovative treatment concepts, EMBO Molecular Medicine 9(9): 1183-1197 (2017). CAR T cells and CAR NK cells are disclosed in U.S. Pat. Nos. 5,712,149; 5,906,936; 6,410,319; 7,446,190; 7,741,465; 7,994,298; 8,975,071; 9,181,527; 9,714,278; 9,518,123; 10,501,519, each of which are hereby incorporated by reference in its entirety. In some embodiments, the CAR T cells comprise a plurality of T cells, wherein at least 80 percent of the T cells of the plurality are CD8+ cells, wherein at least 20%, or at least 30%, or at least 40%, or at least 50%, or at least 60%, or at least 70%, or at least 80%, or at least 90%, or at least 95% of the CD8+ cells express a CAR protein. In some embodiments, the CAR protein comprises one or more antigen recognition moiety and a T cell activation moiety, and optionally wherein the one or more antigen recognition moiety binds to a B cell malignancy-associated antigen. In some embodiments, the CAR protein further comprises a transmembrane domain. In some embodiments, the CAR protein further comprises an intracellular domain of human CD3 ζ chain. In some embodiments, CAR protein comprises, arranged from extracellular to intracellular: one or more antigen recognition moiety, a transmembrane domain, a T cell activation moiety and an intracellular domain of a human CD3 ζ chain. In some embodiments, the CAR T cells express a plurality of CAR proteins, which differ at least with respect to the antigen recognition moiety. In some embodiments, the CAR T cells express two CAR proteins, that differ at least with respect to the antigen recognition moiety. In some embodiments, the CAR T cells express three CAR proteins that differ at least with respect to the antigen recognition moiety. In some embodiments, the CAR T cells express a wild D1L3 enzyme or a variant thereof described herein. In some embodiments, at least 5%, or at least 10%, or at least 20%, or at least 30%, or at least 40%, or at least 50%, or at least 60%, or at least 70%, or at least 80%, or at least 90%, or at least 95% of the CAR T cells express a wild type D1L3 enzyme or any variant D1L3 disclosed herein.

In some embodiments, the CAR T cells are essentially free of CD4+ cells. In some embodiments, the CAR T cells comprise less than 20%, or less than 15%, or less than 10%, or less than 7%, or less than 5%, or less than 3%, or less than 2%, or less than 1%, or less than 0.5%, or less than 0.1% CD4+ cells.

In some embodiments, the T cell is a human T cell, which is derived from a primary human T cell isolated from a human donor, that (i) is modified to functionally impair or to reduce expression of the endogenous T cell receptor (TCR), and (ii) is further modified to express at least one functional exogenous non-TCR that comprises a chimeric receptor comprising a ligand binding domain attached to a signaling domain, wherein said modified primary human T cell is suitable for use in human therapy, and further wherein the isolated primary human T cell modified as in (i) and (ii) elicits no or a reduced graft-versus-host disease (GVHD) response in a histoincompatible human recipient as compared to the GVHD response elicited by a primary human T cell isolated from the same human donor that is only modified as in (ii).

The invention in some aspects provides methods for producing CAR T cells, the method comprising (i) transfecting T cells with a polynucleotide encoding a CAR, and (ii) transfecting T cells with a polynucleotide encoding a wild type D1L3 enzyme or any variant D1L3 disclosed herein. In some embodiments, step (i) is performed before step (ii). In some embodiments, step (ii) is performed before step (i). In some embodiments, step (i) and step (ii) are performed substantially simultaneously. In some embodiments, methods for producing CAR T cells comprise transfecting T cells with a hybrid polynucleotide comprising the polynucleotide encoding the CAR and the polynucleotide encoding a wild type D1L3 enzyme or any variant D1L3 disclosed herein.

In some embodiments, the CAR protein is encoded by a polynucleotide comprising a nucleotide sequence encoding one or more antigen recognition moiety, a second nucleotide sequence encoding a transmembrane domain, a third nucleotide sequence encoding a costimulatory signaling region, and a third nucleotide sequence encoding a zeta chain portion comprising the intracellular domain of human CD3 ζ chain, wherein the polynucleotide expresses each of the one or more antigen recognition moiety, the transmembrane domain, and the costimulatory signaling region, and the zeta chain portion comprising the intracellular domain of human CD3 ζ chain in one single, continuous chain on the surface of the transfected T cells such that the transfected T cells are triggered to activate and/or proliferate and have MHC non-restricted antibody-type specificity when the expressed one or more antigen recognition moiety bind to one or more cognate antigen(s). In some embodiments, the costimulatory signaling region is derived from a protein selected from CD40, OX40, CD28, 4-1BB, FcεRI, FcγRIII, CD27, and CD2.

In some embodiments, the one or more antigen recognition moiety comprises one or more single-chain Fv domains (scFv) comprising a V_{L} linked to a V_{H} of a specific antibody by a flexible linker. In some embodiments, one or more cognate antigen(s) is a B cell malignancy-associated antigen. In some embodiments, the B cell malignancy-associated antigen is selected from B cell maturation antigen (BCMA), CD19, CS 1, CD38, CD138, CD30, CD20, CD25, CD2, CD5, CD7, CD10, CD19, CD22, CD33, CD52, CD56, CD74, CD138, CD317, Her2, VEGFR2, EGFRviii, CXCR4, BCMA, GD2, and GD3. Additional targets and sequences are disclosed in U.S. Patent Publication 2015/0051266, and PCT publication Nos. WO 2013/123061; WO 2015/123642A1, WO 2014/134165A1, the contents of each which is incorporated herein by reference in its entirety. Thus, in some embodiments, the resulting CAR T cells are directed against a B cell malignancy-associated antigen.

In some embodiments, the polynucleotide encoding wild type D1L3 enzyme or any variant D1L3 further comprises a nucleotide sequence encoding a carrier protein, and optionally further comprises a nucleotide sequence encoding any amino acid linker disclosed herein. In some embodiments, the carrier protein is selected from albumin, transferrin, an Fc domain, XTEN, elastin-like protein, and a variant thereof.

In some embodiments, the CAR T cells are derived from autologous T cells. In some embodiments, the CAR T cells are human cells. In some embodiments, the T cells or T-cell progenitors are obtained from an autologous source (e.g., from the same subject as in need of a therapy). In some embodiments, the autologous T cells or T-cell progenitors are obtained from a subject and cultured and expanded in vitro. In some embodiments, the cultured and/or expanded autologous T cells or T-cell progenitors are transfected with a polynucleotide encoding CAR and another polynucleotide encoding wild type D1L3 enzyme or a variant thereof. In some embodiments, the autologous T cells or T-cell progenitors are engineered to express the chimeric T cell receptor and wild type D1L3 enzyme or a variant thereof and are subjected to enrichment and/or purification. In some embodiments, the autologous T cells or T-cell progenitors are engineered to express the chimeric T cell receptor and wild type D1L3 enzyme or a variant thereof, and are optionally cultured in vitro and/or expanded, and administered to a subject in need thereof.

In some embodiments, the CAR T cells are derived from allogeneic T cells, or allogeneic T cell progenitors (e.g., from a different subject as in need of a therapy). In some embodiments, the T cells or T-cell progenitors are obtained from an allogeneic source and subjected to manipulation to alter them to TCR-deficient T cells or T cell progenitors. In some embodiments, the allogeneic T cells or T-cell progenitors are subjected to deletion or downregulation of endogenous T cell receptor. In some embodiments, the allogeneic T cells are modified so that the allogeneic T cells do not express functional T cell receptors (TCRs). Without being bound by theory, it is to be understood that some, or even all, of the TCR subunits/dimers may be expressed on the cell surface, but that the T cell does not express enough functional TCR to induce an undesirable reaction in the host. Without functional TCRs on their surface, the allogeneic T cells fail to mount an undesired immune response to host cells. In some embodiments, the TCR-deficient allogeneic T cells that are modified as disclosed herein, fail to cause graft versus host disease (GVHD), for example, as they cannot recognize the host MHC molecules. In some embodiments, the allogeneic T cells or T-cell progenitors are cultured and expanded in vitro. In some embodiments, the cultured and/or expanded allogeneic T cells or T-cell progenitors, optionally harboring a deletion or downregulation of endogenous T cell receptor, are transfected with a polynucleotide encoding CAR and another polynucleotide encoding wild type D1L3 enzyme or a variant thereof. In some embodiments, the allogeneic T cells or T-cell progenitors are engineered to express the chimeric T cell receptor and wild type D1L3 enzyme or a variant thereof and are subjected to enrichment and/or purification. In some embodiments, the allogeneic T cells or T-cell progenitors are engineered to express the chimeric T cell receptor and wild type D1L3 enzyme or a variant thereof, and are optionally cultured in vitro and/or expanded, and administered to a subject in need thereof.

In some embodiments, the method further comprises enriching and/or purifying CD8+ T cells from the transfected T cells to generate CD8+ CAR T cells. In some embodiments, the T cells are enriched and/or purifying CD8+ cells, e.g., separating CD8+ cells from CD4+ cells, are generally known in the art. In some embodiments, the CD8+ cells are purified by cell sorting. In some embodiments, the CD8+ cells are purified by positive selection. Positive selection can be carried out, for example, by use of antibodies or other CD8- or CD8/CD28-specific binding molecules, which may optionally be coated on paramagnetic beads. In some embodiments, the CD8+ cells are purified by negative selection. Negative selection can be carried out, for example, by expanding peripheral blood mononuclear cells with antibodies directed against non-CD8 cells, for example an anti-CD4 antibody with or without an anti-CD 14 antibody.

In some embodiments, the nucleic acid construct is introduced into the CD8+ cell or T cell by transfection. In some embodiments, the transfection comprises electroporation, nucleofection, cell squeezing, sonoporation, optical transfection, calcium phosphate transfection, and/or particle-based delivery.

In another aspect, the invention provides a method for producing a CAR T cell, the method comprising purifying CD8+ T cells and transducing the cells with a polynucleotide encoding a CAR and a polynucleotide encoding a wild type D1L3 enzyme or a variant thereof, optionally wherein the resulting CD8+ CAR T cells are directed against a B cell malignancy-associated antigen.

In some embodiments, the nucleic acid construct is introduced into the CD8+ cell or T cell by viral transduction. In some embodiments, CD8+ cells are transduced with viral vector comprising a polynucleotide encoding a CAR and a polynucleotide encoding a wild type D1L3 enzyme or a variant thereof. The construction of such vectors is generally known in the art. The viral vector can be, for example, gamma-retroviral vector or lentiviral vector. The CD8 cells can be transduced, for example by incubating the vector with CD8 cells. In some embodiments, the process of transduction is performed more than once on the same cells. In some embodiments of this aspect of the invention, the nucleic acid construct further encodes a marker or enzyme useful for purifying CD8+ T cells and/or CAR T cells, e.g., beta-galactosidase, luciferase, and/or similar proteins known in the art. In some embodiments of this aspect of the invention, a second nucleic acid construct that encodes a marker or enzyme useful for purifying CD8+ T cells and/or CAR T cells, e.g., beta-galactosidase, luciferase, and/or similar proteins known in the art, is introduced into the T cell concomitantly with the nucleic acid construct encoding the CAR.

In some embodiments, peripheral blood mononuclear cells (PBMCs) are obtained from donors by phlebotomy followed by density centrifugation (without limitation, e.g. FICOLL® centrifugation). In some embodiments, CD8+ T cells are purified by positive selection by incubating cells with paramagnetic CD8 microbeads for 15 min at 4° C., loaded on a MACS® Column, and selected by placing the column in a magnetic field. In other embodiments, CD8+ T cells are purified by negative selection by incubating PBMCs with a paramagnetic bead that bind a heterogeneous group of targets corresponding to non-CD8 T-cells (Stemcell Technologies), column loading, magnetic separation, and elutriation of unbound (CD8+) cells. In some embodiments, CD3+ T cells are separated in a similar fashion using CD3 microbeads. In some embodiments, following CD8+ T cell separation, viability of CD8+ T cells is 98%. In some embodiments, over 95% of the total cell population is CD8+ T cells, and over 95% of the CD3+ T cell population is CD8+ T cells. In some embodiments, the purified CD8+ T cells are incubated at 37° C. and then transfected by electroporation with a polynucleotide encoding a CAR and a polynucleotide encoding a wild type D1L3 enzyme or a variant thereof, optionally wherein the antigen recognition moiety of the CAR binds BCMA. In some embodiments, cells are cultured for at least 1 day prior to transfection in the presence of media supplements (without limitation, e.g., anti-CD3 antibody, IL-2, and/or IL-15). In some embodiments, the cells are incubated overnight at 37° C. with 5% CO₂. In some embodiments, the expression of the CAR and binding specificity of the antigen recognition domain (without limitation, e.g. BCMA) are demonstrated by incubating the cells at a 1:50 dilution with a 200 g/ml solution of biotinylated antigen (without limitation, e.g. BCMA) for 30 minutes, washing in a phosphate buffered saline (PBS)-4% bovine serum albumin (BSA) solution, and reincubating with ALEXA FLUOR®-conjugated streptavidin for 15 minutes. In some embodiments, the expression of wild type D1L3 or a variant thereof is confirmed using Western blots or chromatinase activity in the culture supernatants. In some embodiments, dead cells are stained with propidium iodide. In some embodiments, the viability and transfection efficiency are assessed by flow cytometry. In some embodiments, purified, negatively selected CD3+ cells are used as a positive control. In some embodiments, following electroporation, 98% of the CD8+ T cells are viable. In some embodiments, about 70% of the purified cell population is CD8+ T cells that express the CAR, and about 70% cells express the wild type D1L3 enzyme or a variant thereof.

In various embodiments, a subject can be treated with ten or fewer administrations of the cellular therapy, or with four or fewer treatments of the cellular therapy. While T cells (and other host cells) can be engineered to express D1L3 having whole or partial deletions of the C-terminal BD (as described herein), since T cells express PCSK types 3, 5, 6, and 7 (including Furin, PCSK3), expression of wild type D1L3 can be activated by T cells through cleavage within the C-terminal BD. Exemplary T cells include CD4+ T cells or CD8+ T cells (e.g., CTLs). T cells such as gamma delta T cells or Chimeric Antigen Receptor (CAR)-T cells can be employed in some embodiments. In some embodiments, the CAR-T cells are directed against CD19. In some embodiments, D1L3 C-terminal basic domain processing is induced when the T cell is activated (e.g., by activation of the TCR or CAR). Exemplary T cells can be memory T cells, such as (in order of proliferative capacity) T memory stem cells, central memory T cells, or effector memory T cells, or terminally differentiated T cells.

Exemplary T cells for chromatinase cell therapy may recognize (through the TCR or CAR) a cancer-associated antigen, such as a leukemia-associated antigen, or an antigen of a solid tumor. In some embodiments, the T cell recognizes a viral antigen, including but not limited to an oncovirus. Exemplary oncoviruses include Epstein-Barr virus, human papilloma virus, hepatitis B or C virus, human herpes virus (e.g., HSV8), and human T lymphotrophic virus. In some embodiments, the T cell recognizes a coronavirus antigen, such as a coronavirus antigen (e.g., SARS-CoV-2).

In some embodiments, the host cell (e.g., a T cell, B cell, macrophage, or hematopoietic stem cell) secretes D1L3 enzyme having a deletion of at least 12 amino of the C-terminal BD. In some embodiments, the secreted D1L3 enzyme includes enzymes having deletions of one or more of: K291_S305 del, K292_5305 del, K293_5305 del, with respect to SEQ ID NO:4. In these or other embodiments, the polynucleotide encodes a D1L3 enzyme having a deletion of one or more amino acids of the C-terminal BD, such as at least three or at least five amino acids of the C-terminal BD.

In some embodiments, the composition comprises an effective amount of host cells for delivery (e.g., by infusion). An effective amount of host cells to be delivered by the composition can be determined by one of skill in art, and may include, for example, at least about 10⁶ cells, at least about 10⁷ cells, at least about 10⁸ cells, or at least about 10⁹ cells.

The pharmaceutical composition may be formulated for any administration route, including topical, parenteral, or pulmonary administration. In various embodiments, the composition is formulated for intravenous, intradermal, intramuscular, intraperitoneal, intraarticular, subcutaneous, or intraarterial. In some embodiments, the composition is formulated for intravenous or subcutaneous administration.

In other aspects, the present technology provides a method for treating a subject in need of extracellular chromatin degradation, extracellular trap (ET) degradation and/or neutrophil extracellular trap (NET) degradation. The method comprises administering a therapeutically effective amount of the D1L3 enzyme or composition described herein. Exemplary indications where a subject is in need of extracellular chromatin degradation (including ET or NET degradation) are disclosed in PCT/US18/47084, the disclosure of which is hereby incorporated by reference.

Neutrophils, the predominant leukocytes in acute inflammation, generate neutrophil extracellular traps (NETs), lattices of high-molecular weight chromatin filaments decorated with biologically active proteins and peptides, which immobilize bacteria in wounds. Systemic accumulation of NETs harms tissues and organs due to their cytotoxic, proinflammatory, and prothrombotic activity. Indeed, NETs are frequently associated with inflammatory, ischemic, and autoimmune conditions, including Systemic Lupus Erythematosus (SLE).

In various embodiments, the present invention provides a method for treating, preventing, or managing diseases or conditions characterized by the presence or accumulation of NETs. See Jiménez-Alcázar et al., “Host DNases prevent vascular occlusion by neutrophil extracellular traps.” Science 358(6367): 1202-1206 (2017). A number of stimuli, which sometimes contribute to inflammation and/or pathogenesis, induce NETs. These stimuli include phorbol 12-myristate 13-acetate (PMA), a potent mitogen, lipopolysaccharides (LPS), calcium ionophore A23187, the antibiotic nigericin, which also acts as a potassium ionophore, fungi like Candida albicans, and bacteria like Streptococcus agalactiae (a Group B Streptococcus), Klebsiella pneumoniae and viruses like SARS-CoV2. Leppkes et al “Vascular occlusion by neutrophil extracellular traps in COVID-19.” EBioMedicine 58 (2020) 102925 (2020); Claushuis et al., “Role of peptidylargininedeiminase 4 in neutrophil extracellular trap formation and host defense during Klebsiella pneumoniae-induced pneumonia-derived sepsis.” J Immunol. 201:1241-1252 (2018); and Kenny et al., “Diverse stimuli engage different neutrophil extracellular trap pathways.” Elife. 6:e24437 (2017). The diseases or conditions characterized by the presence or accumulation of NETs include, but are not limited to, diseases associated with chronic neutrophilia, neutrophil aggregation and leukostasis, thrombosis and vascular occlusion, ischemia-reperfusion injury, surgical and traumatic tissue injury, an acute or chronic inflammatory reaction or disease, an autoimmune disease, cardiovascular disease, metabolic disease, systemic inflammation, inflammatory diseases of the respiratory tract, renal inflammatory diseases, inflammatory diseases related to transplanted tissue or hematopoetic stem cell transplantation (e.g. graft-versus-host disease), inflammation caused by viral infections (e.g. COVID-19), and cancer (including leukemia). In some embodiments, the present invention provides a method for treating complete or partial vascular or ductal occlusions involving extracellular chromatin, and including NETs in some embodiments.

In some embodiments, the method comprising administering the compositions described herein to the subject. In some embodiments, the subject is at risk of vascular occlusion involving extracellular chromatin, including chromatin released by cancer cells and injured endothelial cells, among others. Thus, in exemplary embodiments, the subject has cancer (e.g., leukemia or solid tumor). In some embodiments, the subject has a hematological cancer selected from multiple myeloma (MM), Hodgkin lymphoma (HL), non-Hodgkin lymphoma (NHL), chronic lymphocytic leukemia (CLL), and acute lymphoblastic leukemia (ALL). In some embodiments, the subject has metastatic cancer.

Subjects receiving therapy for cancer (including but not limited to T cell therapies) are at risk of tumor lysis syndrome and/or cytokine release syndrome, which occurs when tumor cells release their contents (including chromatin) into the bloodstream. Tumor lysis syndrome is a complication during the treatment of cancer, where large amounts of tumor cells are killed at the same time by cancer treatment. Tumor lysis syndrome and/or cytokine release syndrome occurs commonly after the treatment of lymphomas and leukemias. In some embodiments, the therapy described herein treats, reduces, or prevents tumor lysis syndrome.

In still other embodiments, the subject has an inflammatory disease of the respiratory tract, such as the lower respiratory tract. Exemplary diseases include bacterial and viral infections. In some embodiments, the subject has Acute Respiratory Distress Syndrome (ARDS), Acute Lung Injury (ALI), pneumonia, or asthma. Exemplary viral infections in RSV and coronavirus infection (such as SARS, or SARS-CoV-2, e.g., COVID-19 as well as variants thereof).

In still other embodiments, the subject has a disease other than cancer. In some embodiments, the B cell-associated disease is selected from systemic lupus erythematosus (SLE), rheumatoid arthritis, psoriasis, inflammatory bowel disease, celiac sprue, pernicious anemia, scleroderma, Graves disease, Sjogren syndrome, autoimmune hemolytic anemia (AIHA), myasthenia gravis, cryoglobulinemia, thrombotic thrombocytopenic purpura (TTP), allograft rejection (e.g., transplant rejection of lung, kidney, heart, intestine, liver, pancreas, etc.), pemphigus vulgaris, vitiligo, Hashimoto's disease, Addison's disease, reactive arthritis, and type 1 diabetes.

In some embodiments, the subject has SLE. The discovery of NETs raised the speculation that neutrophils may be the predominant source of autoantigens (i.e. dsDNA, chromatin) in SLE (Brinkmann, et al. Neutrophil Extracellular Traps Kill Bacteria. Science, 303(5663): 1532-1545 (2004). Indeed, autoantibodies such as anti-dsDNA, -histone, and -nucleosome antibodies bind to NETs, forming pathological ICs. Hakkim, et al., Impairment of neutrophil extracellular trap degradation is associated with lupus nephritis, Proceedings of the National Academy of Sciences 107: 9813-9818 (2010). The accumulation of NET-IC breaks immune tolerance via activation of adaptive immune cells that lead to the production of autoantibodies against NET components, forming a vicious cycle of inflammation and autoimmunity. Gupta and Kaplan, The role of neutrophils and NETosis in autoimmune and renal diseases. Nat Rev Nephrol. 12(7): 402-13 (2016). Therefore, reducing accumulation of NETs can break the cycle and thus provide an attractive therapeutic strategy for SLE.

In some embodiments, the present invention pertains to the treatment of diseases or conditions characterized by deficiency of D1L3, or a deficiency of D1. In some cases, the subject has a mutation (e.g., a loss of function mutation) in the Dnase1l3 gene or the Dnase1 gene. Such subjects can manifest with an autoimmune disease, such as: systemic lupus erythematosus (SLE), lupus nephritis, scleroderma or systemic sclerosis, rheumatoid arthritis, inflammatory bowel disease, Crohn's disease, ulcerative colitis, and urticarial vasculitis. In some cases, the subject has an acquired inhibitor of D1 (e.g., anti-DNase1-antibody and actin) and/or D1L3 (e.g., anti-Dnase1l3-antibody). Such subjects can also have an autoimmune or inflammatory disease (e.g., SLE, systemic sclerosis).

In some embodiments, the subject has or is at risk of NETs occluding ductal systems. For example, the D1L3 enzymes or compositions disclosed herein can be administered to a subject to treat pancreatitis, cholangitis, conjunctivitis, mastitis, dry eye disease, Stevens-Johnson syndrome, obstructions of vas deferens, or renal diseases.

In some embodiments, the subject has or is at risk of NETs accumulating on endothelial surfaces (e.g. surgical adhesions), the skin (e.g. wounds/scarring), or in synovial joints (e.g. gout and arthritis, e.g., rheumatoid arthritis). The D1L3 enzymes and compositions described herein can be administered to a subject to treat a condition characterized by an accumulation of NETs on an endothelial surface such as, but not limited to, a surgical adhesion.

Other diseases and conditions associated with NETs, which the D1L3 enzymes or compositions disclosed herein may be used to treat or prevent, include: ANCA-associated vasculitis, asthma, chronic obstructive pulmonary disease, a neutrophilic dermatosis, dermatomyositis, burns, cellulitis, meningitis, encephalitis, otitis media, pharyngitis, tonsillitis, pneumonia, endocarditis, cystitis, pyelonephritis, appendicitis, cholecystitis, pancreatitis, uveitis, keratitis, disseminated intravascular coagulation, acute kidney injury, acute respiratory distress syndrome, shock liver, hepatorenal syndrome, myocardial infarction, stroke, ischemic bowel, limb ischemia, testicular torsion, preeclampsia, eclampsia, and solid organ transplant (e.g., kidney, heart, liver, and/or lung transplant). Furthermore, the D1L3 enzymes or compositions disclosed herein can be used to prevent a scar or contracture, e.g., by local application to skin, in an individual at risk thereof, e.g., an individual with a surgical incision, laceration, or burn.

In various embodiments, the subject has a disease that is or has been treated with wild-type Dnases, including D1 and streptodornase. Such diseases or conditions include thrombosis, stroke, sepsis, lung injury, atherosclerosis, viral infection, sickle cell disease, myocardial infarction, ear infection, wound healing, liver injury, endocarditis, liver infection, pancreatitis, primary graft dysfunction, limb ischemia reperfusion, kidney injury, blood clotting, alum-induced inflammation, hepatorenal injury, pleural exudations, hemothorax, intrabiliary blood clots, post pneumatic anemia, ulcers, otolaryngological conditions, oral infections, minor injuries, sinusitis, post-operative rhinoplasties, infertility, bladder catheter, wound cleaning, skin reaction test, pneumococcal meningitis, gout, leg ulcers, cystic fibrosis, Kartegener's syndrome, asthma, lobar atelectasis, chronic bronchitis, bronchiectasis, lupus, primary ciliary dyskinesia, bronchiolitis, empyema, pleural infections, cancer, dry eyes disease, lower respiratory tract infections, chronic hematomas, Alzheimer's disease, and obstructive pulmonary disease.

In some embodiments, the subject has a loss of function mutation in one or both D1L3 genes, and may exhibit symptoms of SLE, or may be further diagnosed with clinical SLE. In such embodiments, the subject may receive systemic therapy with a BD-deleted D1L3 described herein, such as the fusion protein represented by SEQ ID NO:47. In various embodiments, therapeutically effective amounts of the fusion protein represented by SEQ ID NO:47, or other fusion between albumin and a BD-deleted D1L3, are administered once or twice weekly, or once or twice monthly.

In other aspects, the invention provides methods for obtaining cell-free nucleic acid (e.g., cfDNA or cfRNA) from a subject. As disclosed herein, administration of a nuclease with chromatin-degrading activity (such as D1L3 and variants disclosed herein) increases circulating levels of cfDNA. cfDNA can be detected and evaluated for the presence of biomarkers, including tumor biomarkers for cancer screening, diagnosis, prognosis, and disease monitoring.

In various embodiments, the method comprises administering a nuclease with chromatin-degrading activity to a subject, subsequently obtaining a biological sample from the subject, and isolating cell-free nucleic acid from the sample. In accordance with embodiments, the nuclease is suitable for systemic administration (as described herein), such as a eukaryotic nuclease. In some embodiments, the nuclease is selected from DNASE1-LIKE 3 (D1L3), DNASE1 (D1), DNASE1-LIKE 1 (D1L1), DNASE1-LIKE 2 (D1L2), DNASE1-LIKE 3 Isoform 2 (D1L3-2), DNASE2A (D2A), and DNASE2B (D2B) or a variant thereof. In some embodiments, the nuclease is D1L3, and may be a variant disclosed herein, including variants with improved chromatin-degrading activity as compared to the wild-type enzyme. Additional nucleases are disclosed in U.S. Pat. No. 10,696,956; International Publication No. WO 2019/036719; and PCT Application No. PCT/US20/16490, the disclosures of which are hereby incorporated by reference. In some embodiments, the nuclease is D1L3 having a deletion of at least one amino acid of the D1L3 BD. In some embodiments, the D1L3 has a deletion of at least ten amino acids of the BD, or a deletion of the full BD. In some embodiments, the nuclease is D1L3 having a truncation of at least one amino acid of the D1L3 BD. In some embodiments, the D1L3 has a truncation of at least ten amino acids of the BD, or a truncation of the full BD. In some embodiments, the nuclease has a fusion or conjugation to a half-life extension moiety, such as albumin. In some embodiments, the nuclease comprises the amino acid sequence of SEQ ID NO: 47.

In some embodiments, the nuclease is administered to the subject at a subtherapeutic dose. That is, in some embodiments, the nuclease is administered at a dose that does not result in substantial degradation of extracellular chromatin, such as NETs. In some embodiments, the nuclease is administered at a dose that results in fragmentation of extracellular chromatin, such as NETs or chromatin from necrotic tumor cells, but for a short time. For example, in various embodiments, the nuclease (such as D1L3 or the fusion protein of SEQ ID NO:47) is administered at a dose of from about 0.001 mg/kg to about 100 mg/kg. In some embodiments, the dose is least about 0.0025 mg/kg, or at least about 0.01 mg/kg, or at least about 0.1 mg/kg, or at least about 1 mg/kg. In these or other embodiments, the dose is less than about 10 mg/kg, or less than about 5 mg/kg. Exemplary doses are in the range of about 0.1 mg/kg to about 10 mg/kg, or from about 0.01 mg/kg to about 1 mg/kg.

The biological sample in various embodiments is a body fluid sample. In some embodiments, the biological sample is selected from blood, serum, plasma, urine, cerebrospinal fluid, saliva, and amniotic fluid. Protocols for isolating and detecting cf nucleic acids (e.g., cfDNA) from biological fluids are known in the art. See, Stewart C M, et al., The value of cell-free DNA for molecular pathology, J Pathol. 2018 April; 244(5): 616-627. In some embodiments, the biological sample after administration has higher levels of cell free nucleic acids, such as cell-free DNA or cell-free RNA (cfRNA). In some embodiments, the biological sample is pretreated with an agent selected from an anticoagulant and a chelating agent.

In various embodiments, the biological sample is isolated from the subject after about 10 minutes or more of administering the nuclease, or in some embodiments, after about 30 minutes or more, or after about one hour or more, or after about 2 hours or more. In some embodiments, the biological sample is isolated from the subject after about 1 or 2 days. In some embodiments, the biological sample may be isolated in about one hour or less, or about 30 minutes or less. In some embodiments, the sample is isolated from the subject from about 30 minutes to about 4 hours after administration of the nuclease.

Cell free nucleic acid isolated in accordance with the invention, can be evaluated for the presence of disease biomarkers, including for early detection of cancer, as well as for cancer diagnosis or prognosis, including selection or monitoring of therapy. For example, the subject may be suspected of having, at risk of having, or diagnosed as having cancer. In some embodiments, the subject has previously had cancer, or has a genetic predisposition to develop cancer. In some embodiments, the method is employed as part of a routine screening for cancer(s) (e.g., colorectal cancer and adenomas), including early stage cancer. Exemplary cancers for which the invention finds use include colorectal cancer, bladder cancer, brain cancer, breast cancer, pancreatic cancer, liver cancer, endometrial cancer, gastroesophageal cancer, head and neck cancer, hepatocellular cancer, lung cancer (e.g. non-small cell lung cancer and small cell lung cancer), melanoma, bone cancer, ovarian cancer, testicular cancer, prostate cancer, renal cancer, lymphoma, and thyroid cancer. In some embodiments, the cancer is a hematological malignancy.

In some embodiments, the method further comprises evaluating the cell-free DNA, including evaluating the cell free DNA for one or more cancer or drug response biomarkers. In some embodiments, the evaluating comprises one or more of assessment of tumor-associated mutations (including mutational burden), polymorphisms (including SNPs), copy number aberrations (e.g. relative to a reference genome, or parental genome), microsatellite instability, or cancer-specific or cancer-associated changes (e.g., including epigenetic profiles, such as DNA methylation signatures). In some embodiments, the evaluating comprises evaluating histone modifications, including with the use of antibodies specific for epigenetic signatures and nucleosome-protein adducts. Exemplary methods for evaluating epigenetic signatures are disclosed in U.S. Pat. Nos. 10,408,831; 10,184,945; 9,709,569; 9,400,276; 9,222,937; 9,187,780; and 9,128,086, which are hereby incorporated by reference in their entireties.

In some embodiments, the evaluating detects a genetic cancer marker. In some embodiments, the marker is a variant of one or more of the following genes that is associated with cancer: TP53, EGFR, CDKN2A, AKT1, JAK3, TSC1, NF1, CDH1, MML3, CTNNB1, PIK3C2G, GATA1, EPHB1, ESR1, PAK7, FLT4, MAP2K2, KRAS, NRAS, PIK3CA, BRAF, SMAD4, and APC, among others.

In some embodiments, the evaluating is performed by one or more techniques selected from: DNA sequencing, real-time PCR, gel electrophoresis, immunochemistry (antibody-antigen reaction), spectroscopy (e.g. mass spectroscopy), southern blot, polymerase chain reaction (PCR), a recombinase polymerase amplification (RPA), a loop-mediated amplification (LAMP), helicase-dependent amplification (HDA), chromatin immunoprecipitation (ChIP), hybridization (including solution hybridization, capillary hybridization, or hybridization to nucleic acid arrays, e.g. macroarrays, microarrays and high-density oligonucleotide arrays (Gene Chips)), and a combination of any two or more thereof. In some embodiments, the evaluating is performed by DNA amplifying and/or sequencing.

In other aspects, the invention provides a method for treating a subject in need of extracellular chromatin degradation, the method comprising administering a nucleic acid comprising a nucleic acid sequence encoding wild-type DNASE1L3 or a variant thereof. In some embodiments, the nucleic acid is a DNA molecule. In some embodiments, the DNA molecule is a vector or an expression vector, which is optionally, an adeno-associated viral vector (AAV). In other embodiments, the nucleic acid is an mRNA. In some embodiments, the mRNA is a modified mRNA (mmRNA). Without being bound by theory, the nucleic acid is taken up by one or more cells in vivo. In some embodiments, the one or more cells express proteases that cleave one or more positions of the basic domain. In some embodiments, the one or more cells express and secrete a wild-type D1L3 enzyme or a variant thereof. In some embodiments, the one or more cells express and secrete a D1L3 enzyme having a deletion of one or more C-terminal BD amino acids, which leads to enhancement of enzymatic activity.

In other aspects, the invention provides a method for treating a subject in need of extracellular chromatin degradation, the method comprising administering cells that have been manipulated in vitro or ex vivo to express a wild-type DNASE1L3 or a variant thereof. In some embodiments, these method involves (a) transforming a cell in vitro with an exogenous nucleic acid comprising a nucleic acid sequence encoding wild-type DNASE1L3 or a variant thereof, optionally wherein the cell is obtained from the subject; (b) optionally culturing, growing and/or expanding the cell in vitro to generate a progeny of the cell; and (c) administering the cell or the progeny of the cell to the subject. The cell may be as described herein.

In some embodiments, the nucleic acid is an mRNA. In some embodiments, the mRNA is a modified mRNA (mmRNA).

In some embodiments, the variant has a mutation in a D1L3 C-terminal basic domain. In some embodiments, the variant has a deletion of at least one amino acid, or at least 3, or at least 5, or at least 8, or at least 9, or at least 13, or at least 14 or at least 15 C-terminal amino acids of the D1L3 basic domain. In some embodiments, the variant has a truncation of at least one amino acid, or at least 3, or at least 5, or at least 8, or at least 9, or at least 13, or at least 14 or at least 15 C-terminal amino acids of the D1L3 basic domain.

In some embodiments, the deletion retains each of K291, K292, R297, K298, K299, K303, and R304 with respect to SEQ ID NO: 4. In some embodiments, the deletion removes K303 and/or R304 with respect to SEQ ID NO: 4, and wherein the deletion retains K291, K292 and R297, K298 and K299 with respect to SEQ ID NO: 4. In some embodiments, the deletion removes K303 and R304, and one or more of R297, K298 and K299 with respect to SEQ ID NO: 4, and wherein the deletion retains K291 and K292 with respect to SEQ ID NO: 4. In some embodiments, the deletion removes K303, R304, R297, K298 and K299 with respect to SEQ ID NO: 4, and wherein the deletion retains one or both of K291 and/or K292 with respect to SEQ ID NO: 4. In some embodiments, the deletion removes each of K291, K292, R297, K298, K299, K303, and R304 with respect to SEQ ID NO: 4.

In some embodiments, the nuclease has a fusion or conjugation to a half-life extension moiety (e.g., a fusion to a carrier protein). In some embodiments, the half-life extension moiety is an albumin or a Fc domain. In some embodiments, the nuclease is SEQ ID NO: 47. In some embodiments, the wild-type DNASE1L3 or a variant thereof is capable of secretion from eukaryotic cells.

Other aspects and embodiments of the invention will be apparent from the following examples.

### EXAMPLES

### Example 1: Creating Chimeric DNase Enzymes

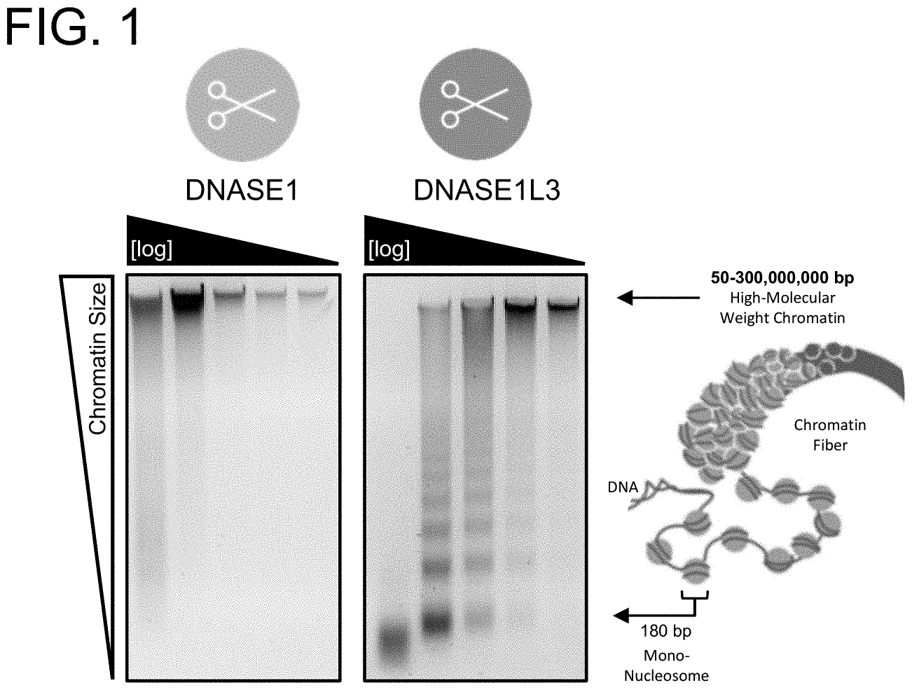

In this Example, chimeric DNase enzymes were created to evaluate the potential to create novel DNase enzymes for therapy against disorders caused by the accumulation of extracellular chromatin, including NETs. To produce variants of D1L3, transient transfection of in vitro expression systems [e.g. Chinese hamster ovary (CHO) cells or HEK293 cells] was used. Enzymatic activity in culture supernatants was characterized using the degradation of high-molecular weight (HMW)-chromatin (i.e. purified nuclei from HEK293 cells) as a readout. In brief, HMW-chromatin was first incubated with the D1L3 variants, followed by DNA isolation and visualization via agarose gel electrophoresis (AGE). As shown in FIG. 1, it was observed that, unlike D1, D1L3 degrades HMW-chromatin (app. 50-300,000,000 base pairs) specifically and efficiently into nucleosomes (app. 180 base pairs), the basic units of chromatin fibers, whereas no such effect was observed in samples with D1.

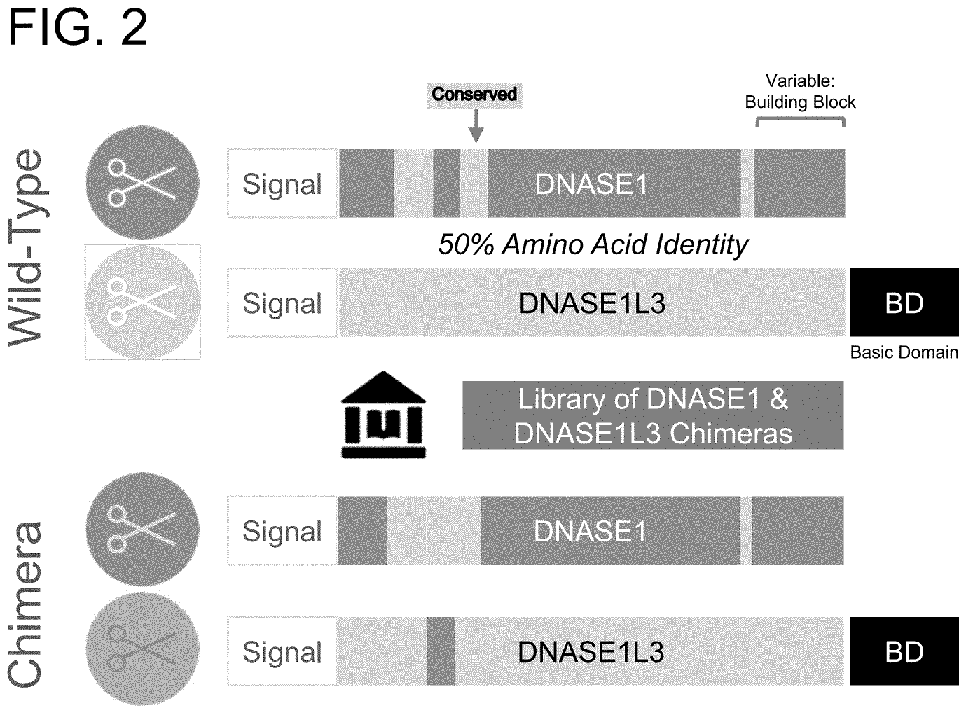

We aimed to identify the regions of D1L3 that are responsible for its chromatin degrading activity. Sequence alignments of human D1 and human D1L3 were performed. The sequence alignments showed that 44% of the amino acids in human D1 and human D1L3 are identical. Without being bound by theory, it was speculated that the capacity of human D1L3 to degrade chromatin is mediated by amino acids that are not present in D1. Thus, only the variable amino acids (56% non-shared amino acids) were mutated to generate D1L3 variants. The method used to transfer enzymatic properties from D1 to D1L3 (building block-technology) is schematically represented in FIG. 2. The following cardinal steps characterize the building block substitution approach:

(1) Provide protein-protein alignment of donor (DNASE1) and recipient DNase (DNASE1L3);

(2) Identify variable amino acid or amino acid sequence for transfer (building block);

(3) Identify conserved amino acids in donor and recipient DNase that are located up and downstream of building blocks, respectively (“anchors”);

(4) Replace the cDNA sequences encoding building block sequences, which are flanked by the C- and N-terminal anchors from a recipient DNase, with the cDNA sequence between the corresponding anchors from donor DNase;

(5) Synthesize a cDNA encoding the chimeric DNase. Prepare an expression vector capable of expressing the chimeric DNase, which harbors the cDNA of the chimeric DNase, operably linked to a promoter, terminator and/or other regulatory sequences of interest.

Chimeric DNase-encoding polynucleotides can be introduced and expressed into a recipient organism/cell of interest, which is preferably deficient in both donor and recipient DNase (e.g. CHO cells or Dnase1^{-/-} Dnase1l3^{-/-} mice).

### Example 2: Biochemical Characterization of the Chimeric Enzymes

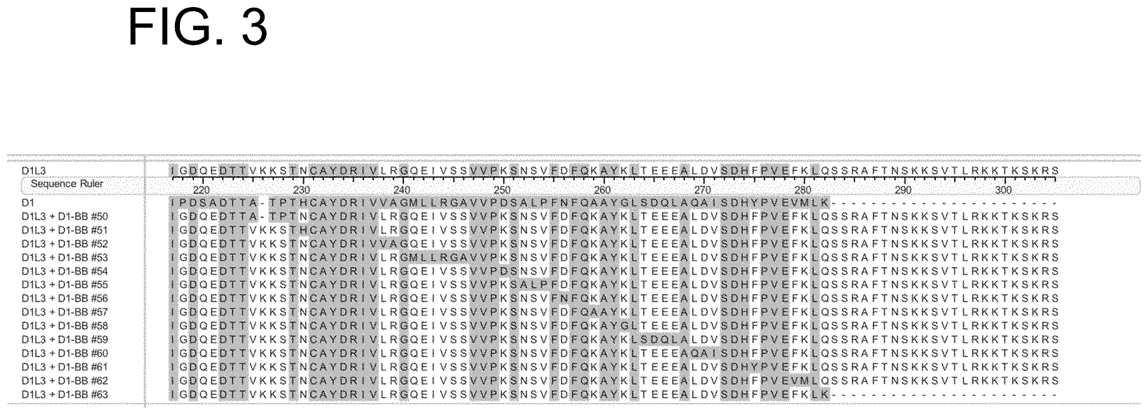

Using the building block substitution approach, 63 D1L3-D1 chimeras were generated (FIG. 3). These chimeras included D1L3-variants with the following building block mutations (BB): M1_A20 delinsMRGMKLLGALLALAALLQGAVS, M21_S25 delinsLKIAA, V28_S30 delinsIQT, E33_S34 delinsET, Q36_145 delinsMSNATLVSYI, K47_K50 delinsQILS, C52Y, 154_M58 delinsIALVQ, 160_K61 delinsVR, S63_I70 delinsSHLTAVGK, M72_K74 delinsLDN, R77_T84 delinsQDAPDT, N86H, V88_I89 delinsVV, S91_R92 delinsEP, N96_T97 delinsNS, Q101R, A103L, L105V, K107_L110 delinsRPDQ, V113_S116 delinsAVDS, H118Y, H120D, Y122_A127 delinsGCEPCGN, V129T, S131N, 135F_136 VdelinsAI, W138R, Q140_H143 delinsFSRF, A145_D148 delinsAVKD, V150A, I152A, T156_T157 delinsAA, E159_S161 delinsGDA, K163A, E167A, V169_E170 delinsYD, T173L, K176_R178 delinsQEK, K180_A181 delinsGL, N183_F186 delinsDVML, P198_A201 delinsRPSQ, K203_N204 delinsSS, R208W, D210S, R212T, V214Q, G218P, Q220_E221 delinsSA, V225_S228 delinsATP, N230H, L238_R239 delinsVA, Q241_S246 delinsMLLRGA, K250D, N252_V254 delinsALP, D256N, K259_A260 delinsAA, K262G, T264_E267 delinsSDQL, L269_V271 delinsQAI, F275Y, F279_K280 delinsVM, and Q282_S305 delinsK.

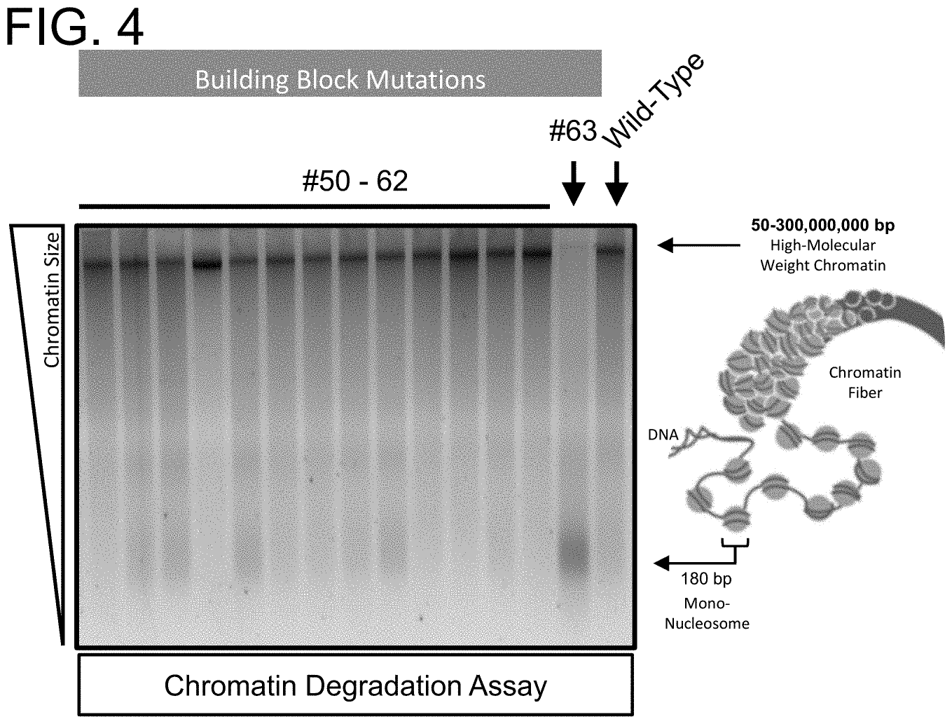

The D1L3-variants were transiently expressed in CHO cells and culture supernatants were screened for the activity to degrade high molecular weight (HMW)-chromatin. The reaction mixtures of HMW-chromatin degradation assay were examined by agarose gel electrophoresis (AGE) to assess the activity of the D1L3-D1 chimera. As shown in FIG. 4, these assays led to the identification of Q282_S305 delinsK (i.e. BB #63), which confers hyperactivity on D1L3.

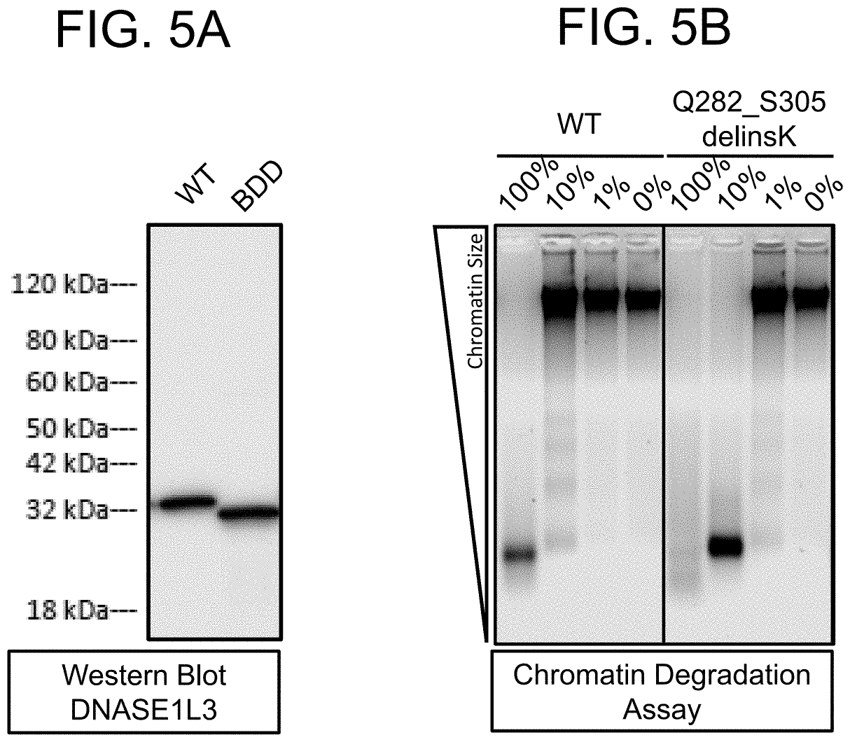

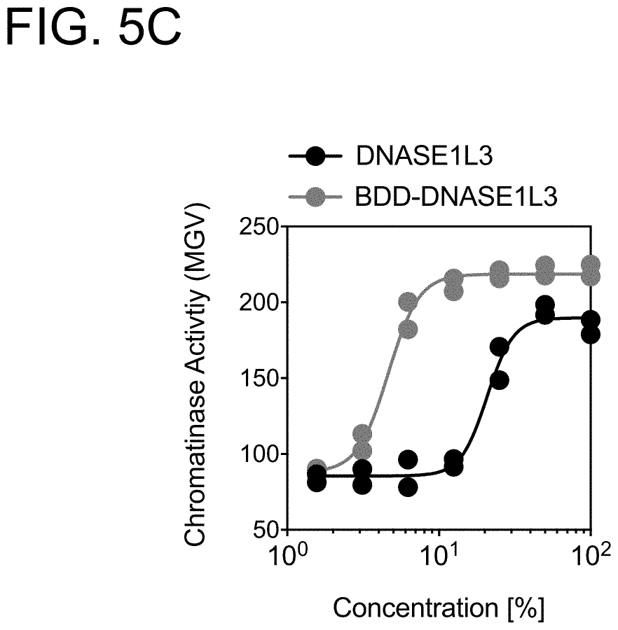

The mutation Q282_S305 delinsK causes a complete deletion of the BD domain and the substitution of Q282 (glutamine at position 282) of D1L3 with a K (Lysine). As shown in FIG. 5A, the deletion of the C-terminal BD was confirmed using Western Blotting of culture supernatants using a monoclonal antibody that targets MGDFNAGCSYV (SEQ ID NO:42) (Anti-D1/D1L3), a peptide sequence that is shared by D1 and D1L3. The data further illustrated similar protein concentration of wild-type and mutated D1L3, indicating a lack of toxicity or altered protease sensitivity of the Q282_S305 delinsK mutant D1L3 protein in the expression system (FIG. 5A).

To compare the enzymatic activity of wild type and Q282_S305 delinsK mutant D1L3 proteins, a titration experiment was performed: high molecular weight chromatin was digested with increasing amounts of the enzymes, and reaction products were resolved by agarose gel electrophoresis (AGE). As shown in FIG. 5B, titration of culture supernatants showed that BD-deleted D1L3 had approximately 5- to 10-fold higher activity to degrade HMW-chromatin. The extent of chromatinase activity of wild type and Q282_S305 delinsK mutant D1L3 proteins was calculated based on the quantitation of degraded chromatin (FIG. 5C). Collectively, the data suggest that the BD domain of D1L3 is not required for chromatin degradation, but rather suppresses enzymatic activity.

### Example 3: Comparison of Different C-Terminal Truncation Mutants

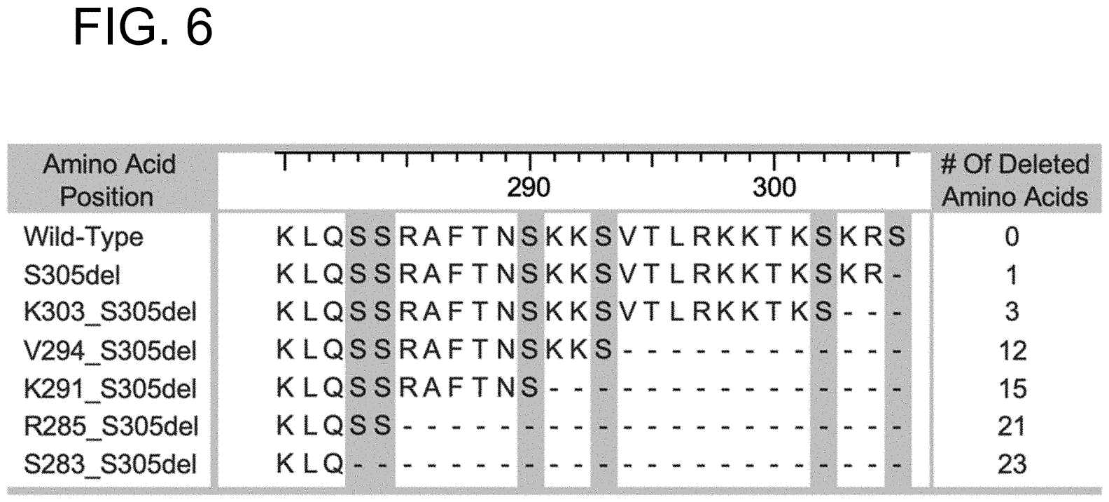

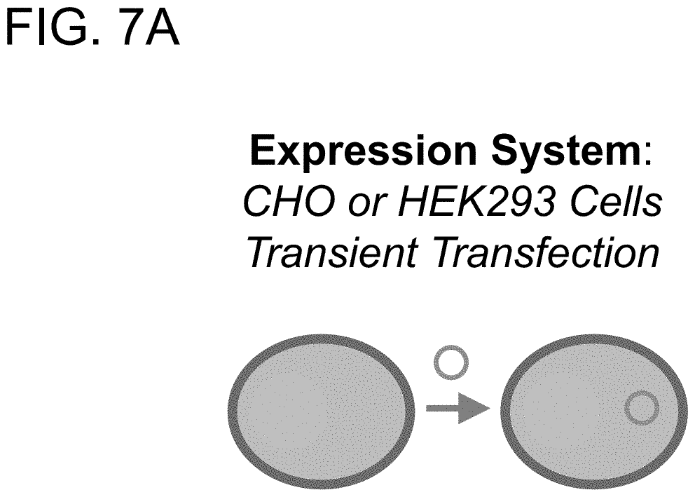

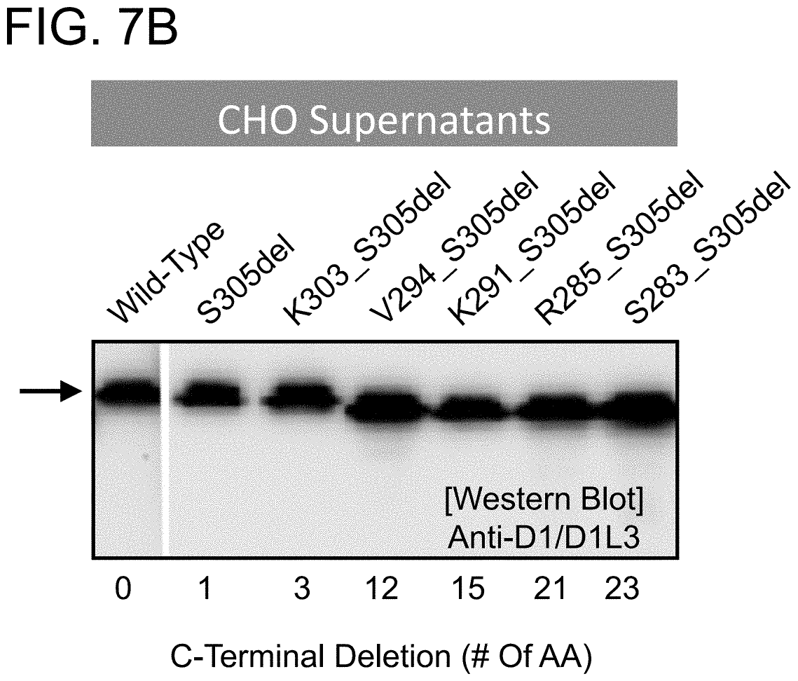

Since the Q282_S305 delinsK mutation lacking the BD domain showed approximately 5 to 10-fold higher chromatinase activity compared to wild type D1L3 protein, the effect of extent of C-terminal deletion was evaluated. Whether full or partial truncation of BD of D1L3 is required to enable chromatin degradation was explored. Deletion mutants S305 del, K303_S305 del, V294_S305 del, K291_S305 del, R285_S305 del, and 5283_S305 del, which lack one, 3, 12, 15, 21, and 23 C-terminal amino acids, respectively were designed (FIG. 6). CHO and HEK293 cells were transiently transfected with vectors expressing wild-type and truncation mutants (FIG. 7A). Culture supernatants containing D1L3 and the variants were collected and tested by Western Blot using the aforementioned anti-D1/D1L3 antibody. As shown in FIG. 7B, wild-type D1L3 as well as variants of smaller molecular weight could be detected. The observed mobility shift, inter alia, confirmed the deletion of BD. Furthermore, the western blots showed that all C-terminal truncation mutants and the wild-type D1L3 exhibited similar protein expression levels.

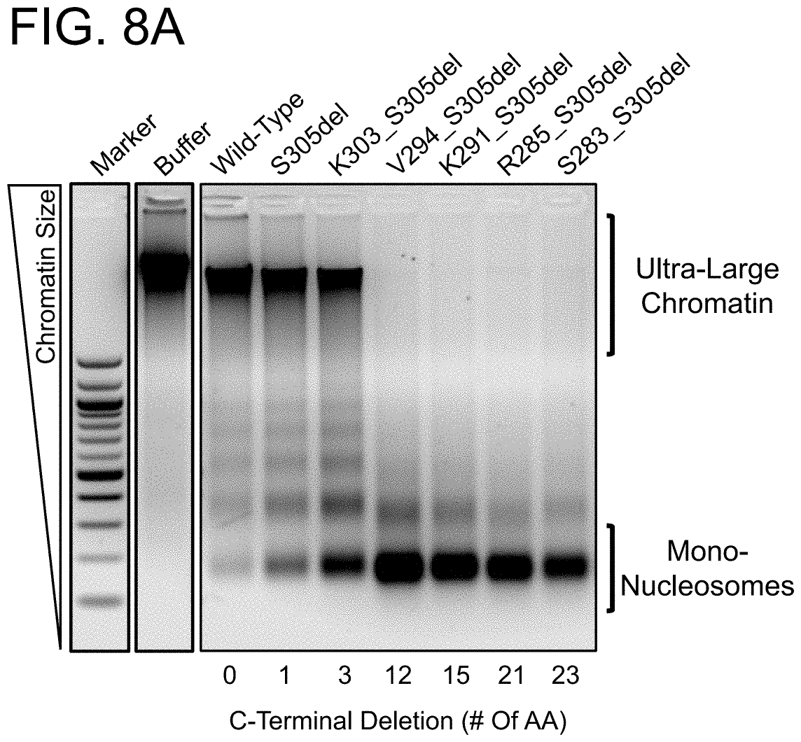

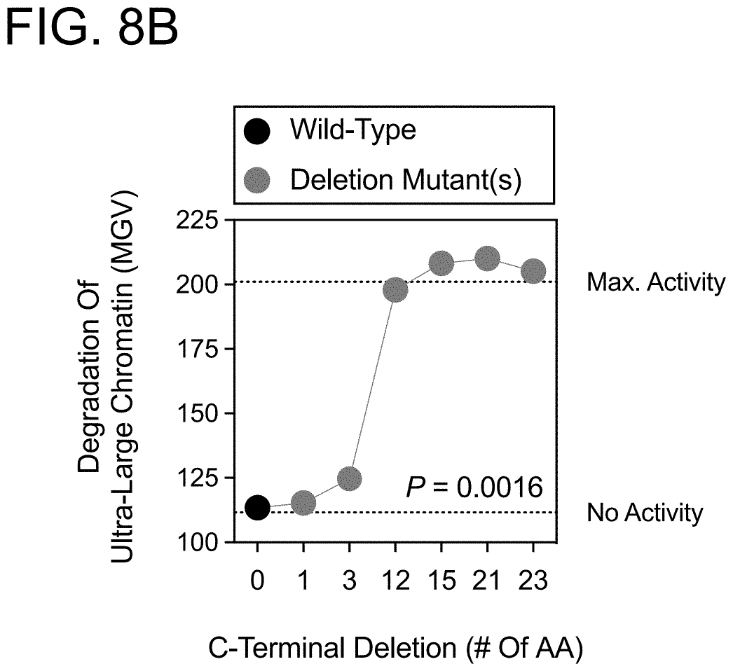

The effect of deleting all or part of the BD on chromatin degrading activity was determined by incubating culture supernatants with intact chromatin from isolated nuclei. In the first set of experiments, the culture supernatants of cells expressing wild-type and truncation mutants were diluted 10-fold with incubation buffer and then incubated with high molecular weight chromatin. Analysis of DNA fragmentation by AGE revealed that deletion of 3 or less amino acids caused only a minor increase in enzymatic activity, whereas the deletion of 12 or more amino acids strongly accelerated the degradation of ultra-large chromatin into mono-nucleosomes (FIG. 8A). To quantify chromatin degrading activity, the concentration of ultra-large chromatin, defined a as DNA-fragments of >1,500 base pairs, was determined using image analysis of agarose gel electrophoresis (AGE). As shown in FIG. 8B, this analysis confirmed the correlation of BD-deletion and enzymatic activity.

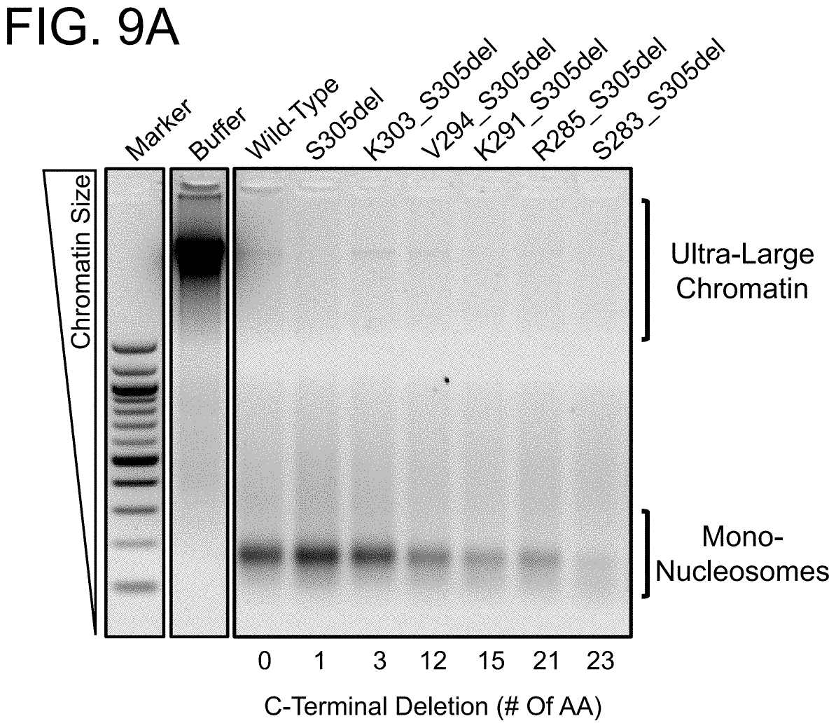

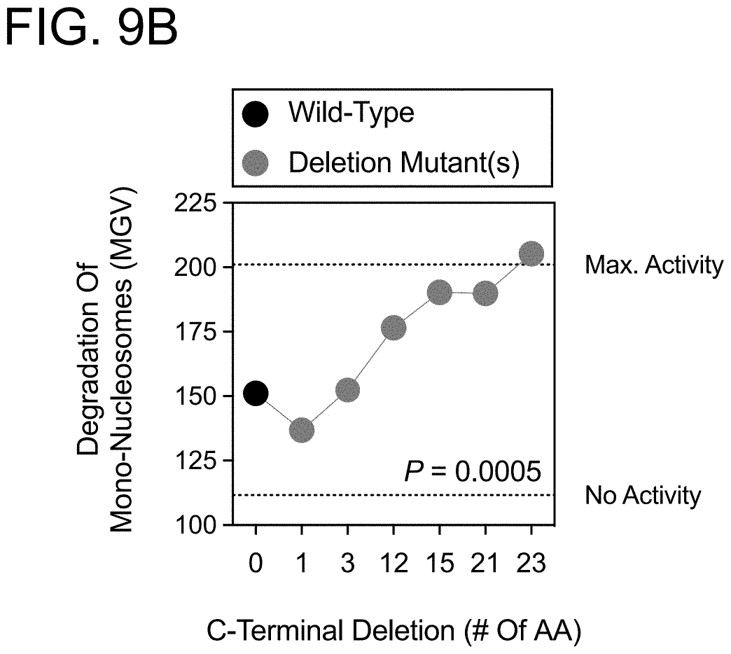

In a second set of experiments, undiluted culture supernatants of cells expressing wild-type and truncation mutants were mixed with intact chromatin from isolated nuclei. Under these conditions, ultra-large chromatin was completely degraded into mono-nucleosomes by D1L3 samples (FIG. 9A). Next, mono-nucleosomes, defined as DNA-fragments between 100 and 300 base pairs, were quantified using image analysis. As shown in FIG. 9B, a linear correlation was observed between the deletion of C-terminal amino acids and the activity of D1L3 to degrade mono-nucleosomes. These data showed that enzymatic activity for degrading mononucleosomes increased with increasing length of deletion from three C-terminal amino acids to 23 C-terminal amino acids (FIG. 9B). Taken together, the data illustrate that the entire BD of D1L3 has distinct inhibitory effects on degradation of chromatin (including ultra-large chromatin). These data also illustrate that the BD of D1L3 has inhibitory effects on degradation of mono-nucleosomes.

### Example 4: The Paired Basic Amino Acid Cleaving Enzyme Furin Regulates D1L3

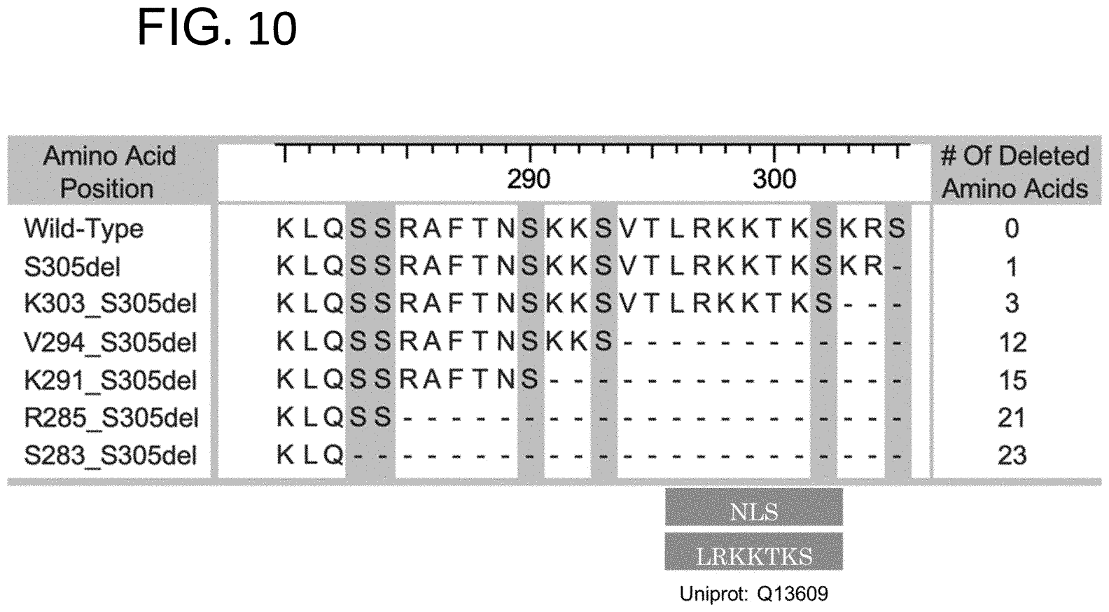

The BD domain contains an NLS and three paired basic amino acids that are potentially responsible for the inhibitory effects on enzymatic activity (FIG. 10). The NLS is located between amino acids L286 to S302 [LRKKTKS; Reference: Q13609 (Uniprot)] and therefore absent in all identified hyperactive D1L3 mutants that lack 12 amino acids more (FIGS. 8A, 8B, 9A, 9B and 10).

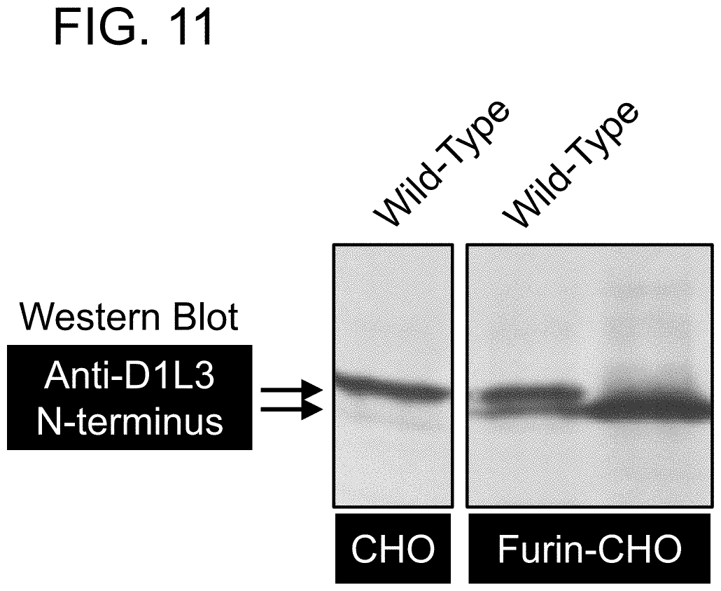

Without being bound by theory, it was hypothesized that three sets of paired basic amino acids are K291/K292, R297/K298/K299, and K303/R304 and may serve as proteolytic cleavage sites of the Paired Basic Amino Acid Cleaving Enzyme (PACE). Furin is a well-characterized PACE, which is involved in the maturation of pro-enzymes. To test the possibility of furin generating active D1L3, and to understand the possible role of furin in activation of chromatinase activity of D1L3, furin-overexpressing CHO cells were transiently transfected with wild-type and BD-deleted D1L3 (S283_S305 del mutant). CHO cells without overexpression of furin were included as control. Culture supernatants were collected and tested by western blot using an antibody that targets the N-terminus of D1L3. As shown in FIG. 11, two bands of D1L3 were detected in furin-overexpressing CHO cells. The top band corresponded to D1L3 expressed by CHO cells without overexpression of furin, whereas the lower band corresponded to the BD-deleted D1L3 (FIG. 11). The data suggest that furin and/or other proteases may directly or indirectly delete the BD and thus cause the maturation of D1L3 into its enzymatically active form.

### Example 5: Single Amino Acid Deletion Generated D1L3 with Chromatin and DNA-Degrading Activity

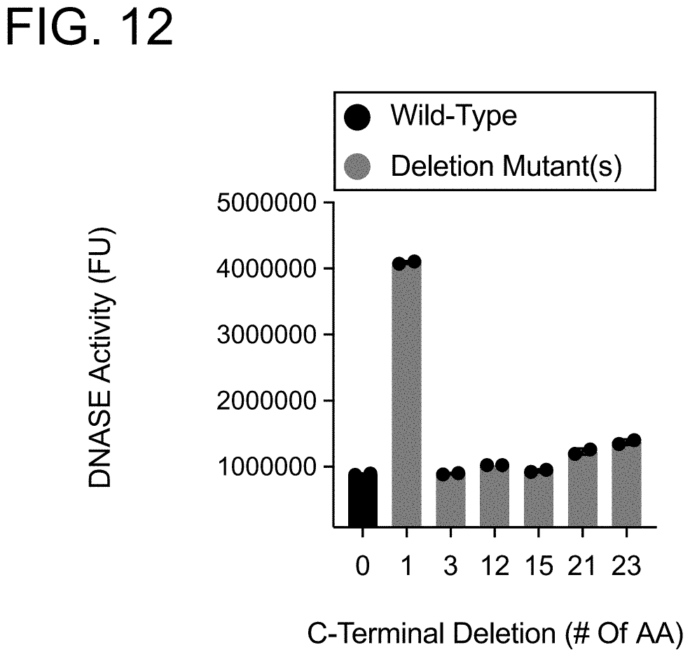

Further, we tested the activity of the BD-deleted D1L3 mutants to degrade protein-free DNA. In brief, the D1L3-variants were transiently expressed in CHO cells and culture supernatants were incubated with a commercially available DNA-probe, which becomes fluorescent upon cleavage by a DNASE, i.e. DNASE Alert. Surprisingly, we observed a robust increase in DNASE activity upon deletion of the C-terminal serine residue (e.g., S305 of SEQ ID NO:4) (FIG. 12). The deletion of a single amino acid—the C-terminal serine (S305)—generated a DNASE1L3 variant that has chromatinase activity and high DNASE activity (SEQ ID NO: 22).

### Example 6: Heterologous Expression of Wild-Type D1L3

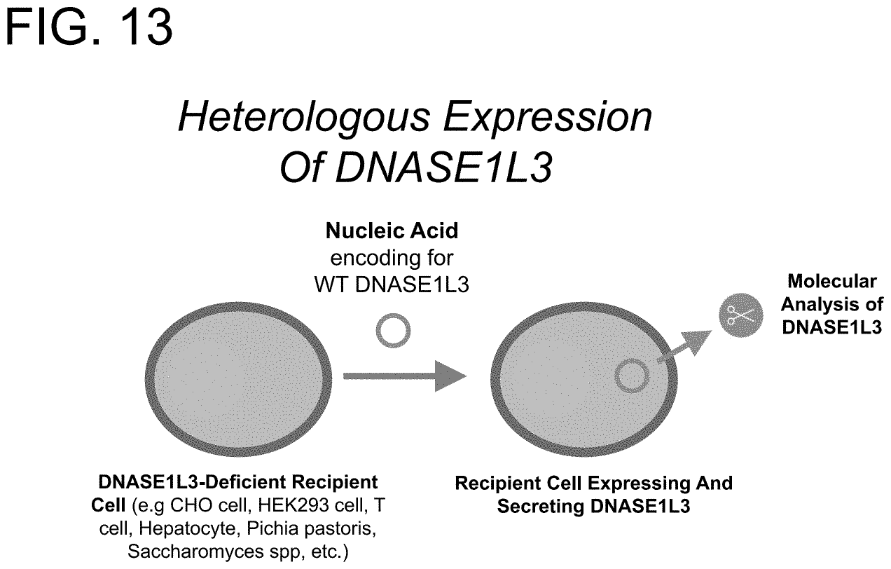

We analyzed molecular modifications of the C-terminal BD of D1L3 following the heterologous expression of wild-type D1L3 (FIG. 13).

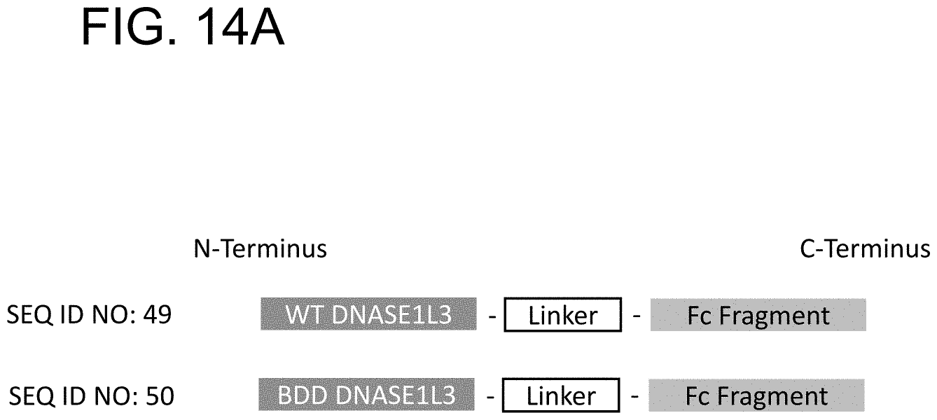

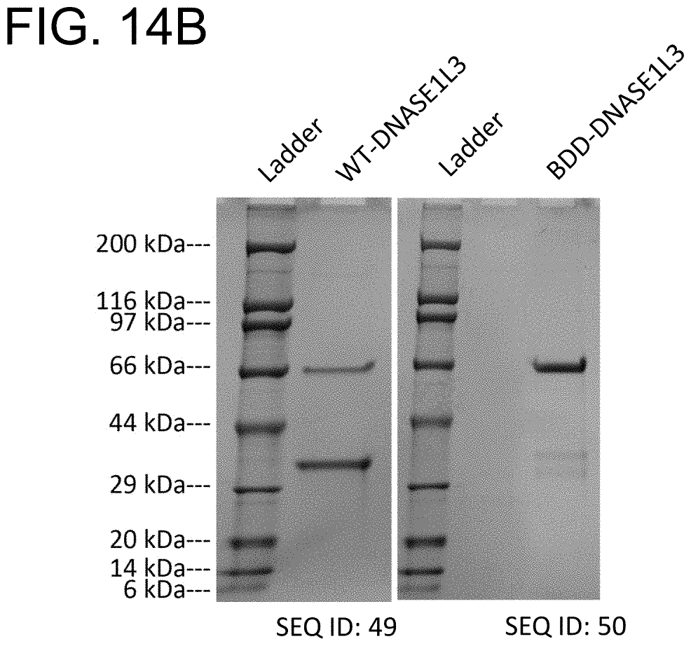

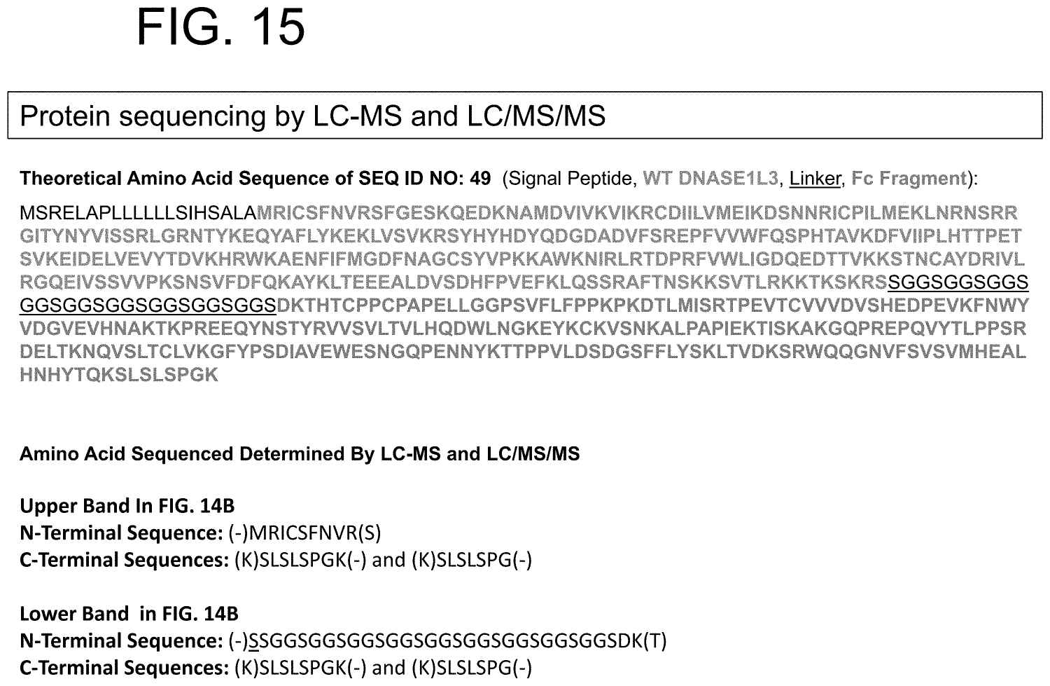

In the first study, we expressed a wild-type D1L3 that was linked to the Fc fragment via a flexible glycine-serine linker in CHO cells (FIG. 14A). As a control, we used an Fc fusion protein of a Basic Domain Deleted-D1L3. CHO cells were transiently transfected with either DNA construct. We collected supernatants and purified the Fc fusion proteins using protein A. Analysis of purified proteins by SDS-PAGE showed bands of approximately 65-70 kDa, which reflects the molecular weight of the full-length proteins (FIG. 14B). An additional lower molecular weight band (between 29-44 kDa) was observed in proteins purified from CHO cells expressing the WT-DNASE1L3 construct. Using LC-MS and LC/MS/MS, we identified that the N- and C-terminal amino acid sequence of the upper band corresponds to mature DNASE1L3 and Fc fragment, respectively. As seen by others, clipping of the C-terminal lysine residue of the Fc fragment was observed. Surprisingly, analysis of the lower band showed that the N-terminus of the protein contained the serine from the C-terminus of DNASE1L3, i.e. Serine 305 in SEQ ID NO: 4 (FIG. 15). Collectively these data suggest that the expression of wild-type DNASE1L3 leads to the secretion of clipping of the C-terminal seine residue.

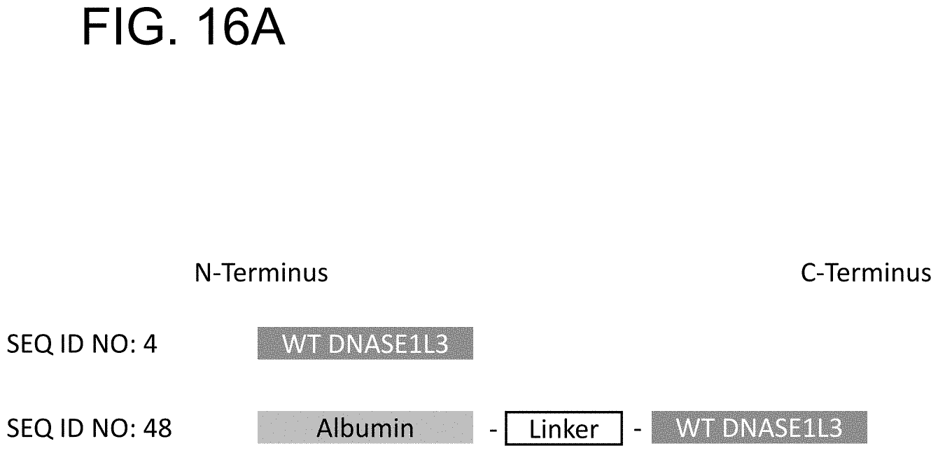

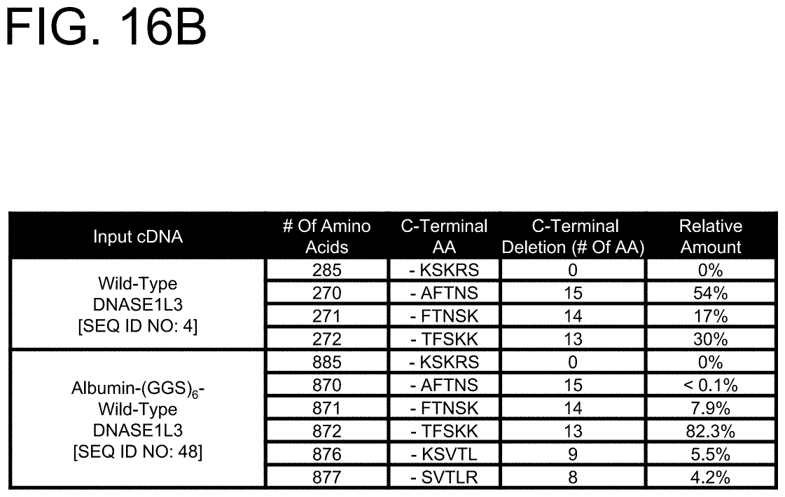

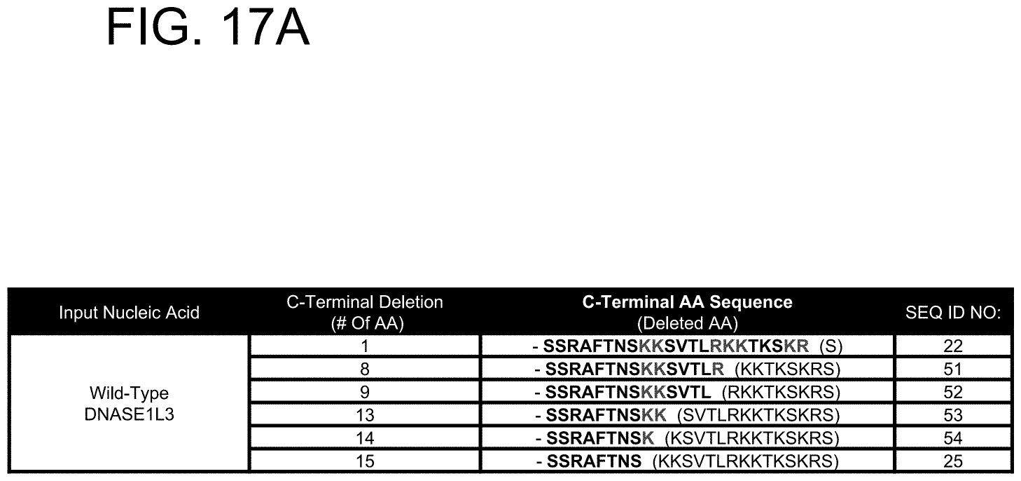

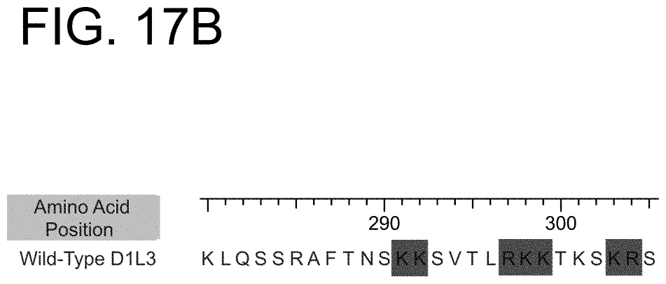

In the second study, we expressed a wild-type D1L3 and a D1L3 linked at the N-terminus to albumin via a flexible glycine-serine linker in Pichia pastoris (FIG. 16A). We collected fermentation supernatants and purified the proteins using affinity chromatography. Surprisingly, we observed no full-length D1L3 protein with either expression construct, but various degrees of C-terminal truncations as identified by LC-MS and LC/MS/MS. The expression of wild-type D1L3 produced deletions of 13, 14, and 15 amino acids. Additional proteins with deletions of 8 and 9 amino acids were detected in samples of the wild-type D1L3 albumin fusion protein (FIG. 16B). In summary, the heterologous expression of wild-type D1L3 leads to the secretion of D1L3 variants with distinct deletions in the C-terminal basic domain. Importantly, the deletions occur after, within, or before the three clusters of basic amino acids (FIG. 17A). In further studies, we expressed a wild-type D1L3 linked at the C-terminus to albumin via a flexible glycine-serine linker in Pichia pastoris and observed two proteins with a molecular weight similar to D1L3 and albumin, indicating the proteolytic cleavage of the fusion protein in the linker region that contains the BD. Collectively, our data identified the K291/K292, R297/K298/K299, and K303/R304 in SEQ ID NO: 4 as sites for post-translational modifications of wild-type DNASE1L3 (FIG. 17B).

### Example 7: Enrichment of Cell-Free DNA in Serum after Intravenous Injection of DNASE1L3 Variant

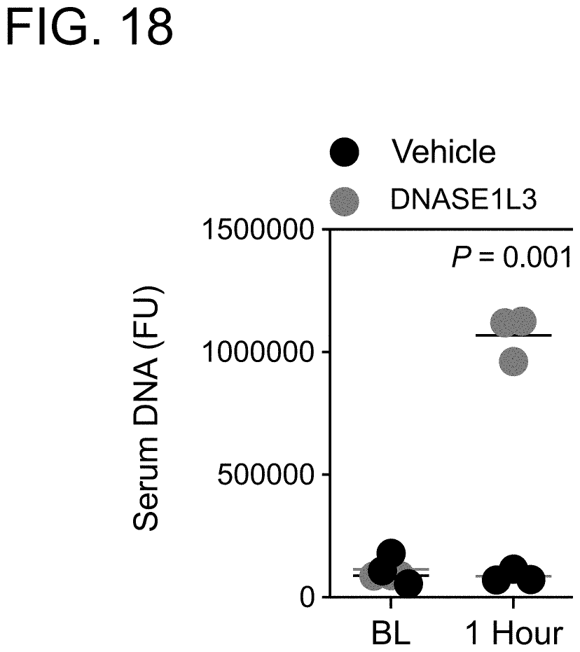

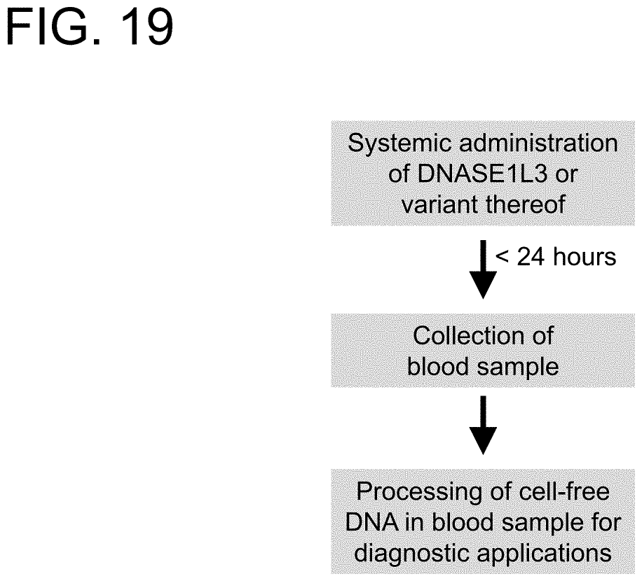

The following example evaluates whether chromatin degradation products are enriched in serum after the systemic delivery of recombinant DNASE1L3. In short, liver necrosis was induced in Dnase1^{-/-} Dnase1l3^{-/-} mice by injecting acetaminophen (APAP), a well-known hepatotoxic agent, followed by the intravenous injection of a recombinant DNASE1L3 variant with high chromatin degrading activity. It was hypothesized that DNASE1L3 cleaves high-molecular weight chromatin that is exposed by necrotic hepatocytes into fragments that are detectable as cell-free DNA in serum. Indeed, as shown in FIG. 18, strongly increased levels of cell-free DNA was detected in serum one hour after injection of recombinant DNASE1L3. Specifically, injection of recombinant DNASE1L3 led to about 10× increase in serum DNA (FIG. 18). In conclusion, the study shows that injection of DNASE1L3 can enrich serum with cell-free DNA from necrotic cells. Consequently, our finding can improve diagnostic applications that use epigenetic or sequence information of cell-free DNA for diagnostics. We propose to inject a subject in need for diagnostic testing of its cell-free DNA with a subtherapeutic dose of recombinant DNASE1L3 shortly before blood collection. The invention in these embodiments has particular value for cancer screening and evaluation.

Wild-Type Human DNASES
DNASE1 (NP_005212.2): Signal Peptide, Mature Protein:
SEQ ID NO: 1
MRGMKLLGALLALAALLQGAVSLKIAAFNIQTFGETKMSNATLVSYIVQILSRYDIAL
VQEVRDSHLTAVGKLLDNLNQDAPDTYHYVVSEPLGRNSYKERYLFVYRPDQVSAVDS
YYYDDGCEPCGNDTFNREPAIVRFFSRFTEVREFAIVPLHAAPGDAVAEIDALYDVYL
DVQEKWGLEDVMLMGDFNAGCSYVRPSQWSSIRLWTSPTFQWLIPDSADTTATPTHCA
YDRIVVAGMLLRGAVVPDSALPFNFQAAYGLSDQLAQAISDHYPVEVMLK
DNASE1-LIKE 1 (NP_006721.1): Signal Peptide; Mature Protein:
SEQ ID NO: 2
MHYPTALLFLILANGAQAFRICAFNAQRLTLAKVAREQVMDTLVRILARCDIMVLQEV
VDSSGSAIPLLLRELNRFDGSGPYSTLSSPQLGRSTYMETYVYFYRSHKTQVLSSYVY
NDEDDVFAREPFVAQFSLPSNVLPSLVLVPLHTTPKAVEKELNALYDVFLEVSQHWQS
KDVILLGDFNADCASLTKKRLDKLELRTEPGFHWVIADGEDTTVRASTHCTYDRVVLH
GERCRSLLHTAAAFDFPTSFQLTEEEALNISDHYPVEVELKLSQAHSVQPLSLTVLLL
LSLLSPQLCPAA
DNASE1-LIKE 2 (NP_001365.1): Signal Peptide, Mature Protein:
SEQ ID NO: 3
MGGPRALLAALWALEAAGTAALRIGAFNIQSFGDSKVSDPACGS11AKILAGYDLALV
QEVRDPDLSAVSALMEQINSVSEHEYSFVSSQPLGRDQYKEMYLFVYRKDAVSVVDTY
LYPDPEDVFSREPFVVKFSAPGTGERAPPLPSRRALTPPPLPAAAQNLVLIPLHAAPH
QAVAEIDALYDVYLDVIDKWGTDDMLFLGDFNADCSYVRAQDWAAIRLRSSEVFKWLI
PDSADTTVGNSDCAYDRIVACGARLRRSLKPQSATVHDFQEEFGLDQTQALAISDHFP
VEVTLKFHR
DNASE1-LIKE 3; Isoform 1 (NP_004935.1): Signal Peptide,
Mature Protein:
SEQ ID NO: 4
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKSVTLRKKTKSKRS
DNASE1-LIKE 3, Isoform 2 (NP_001243489.1): Signal Peptide;
Mature Protein:
SEQ ID NO: 5
MSRELAPLLLLLLSIHSALAMRICSFNVRSFGESKQEDKNAMDVIVKVIKRCDIILVM
EIKDSNNRICPILMEKLNREKLVSVKRSYHYHDYQDGDADVFSREPFVVWFQSPHTAV
KDFVIIPLHTTPETSVKEIDELVEVYTDVKHRWKAENFIFMGDFNAGCSYVPKKAWKN
IRLRTDPRFVWLIGDQEDTTVKKSTNCAYDRIVLRGQEIVSSVVPKSNSVFDFQKAYK
LTEEEALDVSDHFPVEFKLQSSRAFTNSKKSVTLRKKTKSKRS
DNASE2A (O00115): Signal Peptide; Mature Protein:
SEQ ID NO: 6
MIPLLLAALLCVPAGALTCYGDSGQPVDWFVVYKLPALRGSGEAAQRGLQYKYLDESS
GGWRDGRALINSPEGAVGRSLQPLYRSNTSQLAFLLYNDQPPQPSKAQDSSMRGHTKG
VLLLDHDGGFWLVHSVPNFPPPASSAAYSWPHSACTYGQTLLCVSFPFAQFSKMGKQL
TYTYPWVYNYQLEGIFAQEFPDLENVVKGHHVSQEPWNSSITLTSQAGAVFQSFAKFS
KFGDDLYSGWLAAALGTNLQVQFWHKTVGILPSNCSDIWQVLNVNQIAFPGPAGPSFN
STEDHSKWCVSPKGPWTCVGDMNRNQGEEQRGGGTLCAQLPALWKAFQPLVKNYQPCN
GMARKPSRAYKI
DNASE2B (Q8WZ79): Signal Peptide; Mature Protein:
SEQ ID NO: 7
MKQKMMARLLRTSFALLFLGLFGVLGAATISCRNEEGKAVDWFTFYKLPKRQNKESGE
TGLEYLYLDSTTRSWRKSEQLMNDTKSVLGRTLQQLYEAYASKSNNTAYLIYNDGVPK
PVNYSRKYGHTKGLLLWNRVQGFWLIHSIPQFPPIPEEGYDYPPTGRRNGQSGICITF
KYNQYEAIDSQLLVCNPNVYSCSIPATFHQELIHMPQLCTRASSSEIPGRLLTTLQSA
QGQKFLHFAKSDSFLDDIFAAWMAQRLKTHLLTETWQRKRQELPSNCSLPYHVYNIKA
IKLSRHSYFSSYQDHAKWCISQKGTKNRWTCIGDLNRSPHQAFRSGGFICTQNWQIYQ
AFQGLVLYYESCK
Chimeras of Human DNASE1L3 and DNASE1
D1L3 + D1-BB#50: Signal Peptide; Mature Protein (Building
block transferred from D1)
SEQ ID NO: 8
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFWWFQSPHTAVKDFVIIPLHTTPETSVKEIDELVEVYTD
VKHRWKAENFIFMGDFNAGCSYVPKKAWKNIRLRTDPRFVWLIGDQEDTTATPTNCAY
DRIVLRGQEIVSSVVPKSNSVFDFQKAYKLTEEEALDVSDHFPVEFKLQSSRAFTNSK
KSVTLRKKTKSKRS
D1L3 + D1-BB#51: Signal Peptide; Mature Protein (Building
block transferred from D1)
SEQ ID NO: 9
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTHCA
YDRIVLRGQEIVSSVVPKSNSVFDFQKAYKLTEEEALDVSDHFPVEFKLQSSRAFTNS
KKSVTLRKKTKSKRS
D1L3 + D1-BB#52: Signal Peptide; Mature Protein (Building
block transferred from D1)
SEQ ID NO: 10
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVVAGQEIVSSVVPKSNSVFDFQKAYKLTEEEALDVSDHFPVEFKLQSSRAFTNS
KKSVTLRKKTKSKRS
D1L3 + D1-BB#53: Signal Peptide; Mature Protein (Building
block transferred from D1)
SEQ ID NO: 11
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGMLLRGAVVPKSNSVFDFQKAYKLTEEEALDVSDHFPVEFKLQSSRAFTNS
KKSVTLRKKTKSKRS
D1L3 + D1-BB#54: Signal Peptide; Mature Protein (Building
block transferred from D1)
SEQ ID NO: 12
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPDSNSVFDFQKAYKLTEEEALDVSDHFPVEFKLQSSRAFTNS
KKSVTLRKKTKSKRS
D1L3 + D1-BB#55: Signal Peptide; Mature Protein (Building
block transferred from D1)
SEQ ID NO: 13
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNALPDFQKAYKLTEEEALDVSDHFPVEFKLQSSRAFTNS
KKSVTLRKKTKSKRS
D1L3 + D1-BB#56: Signal Peptide; Mature Protein (Building
block transferred from D1)
SEQ ID NO: 14
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFNFQKAYKLTEEEALDVSDHFPVEFKLQSSRAFTNS
KKSVTLRKKTKSKRS
D1L3 + D1-BB#57: Signal Peptide; Mature Protein (Building
block transferred from D1)
SEQ ID NO: 15
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQAAYKLTEEEALDVSDHFPVEFKLQSSRAFTNS
KKSVTLRKKTKSKRS
D1L3 + D1-BB#58: Signal Peptide; Mature Protein (Building
block transferred from D1)
SEQ ID NO: 16
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYGLTEEEALDVSDHFPVEFKLQSSRAFTNS
KKSVTLRKKTKSKRS
D1L3 + D1-BB#59: Signal Peptide; Mature Protein (Building
block transferred from D1)
SEQ ID NO: 17
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLSDQLALDVSDHFPVEFKLQSSRAFTNS
KKSVTLRKKTKSKRS
D1L3 + D1-BB#60: Signal Peptide; Mature Protein (Building
block transferred from D1)
SEQ ID NO: 18
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEAQAISDHFPVEFKLQSSRAFTNS
KKSVTLRKKTKSKRS
D1L3 + D1-BB#61: Signal Peptide; Mature Protein (Building
block transferred from D1)
SEQ ID NO: 19
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHYPVEFKLQSSRAFTNS
KKSVTLRKKTKSKRS
D1L3 + D1-BB#62: Signal Peptide; Mature Protein (Building
block transferred from D1)
SEQ ID NO: 20
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEVMLQSSRAFTNS
KKSVTLRKKTKSKRS
D1L3 + D1-BB#63: Signal Peptide; Mature Protein (Building
block transferred from D1)
SEQ ID NO: 21
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLK
C-terminal deletion mutants of Human DNASE1L3
S305del: Signal Peptide; Mature Protein:
SEQ ID NO: 22
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKSVTLRKKTKSKR
K303_S305del: Signal Peptide; Mature Protein:
SEQ ID NO: 23
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKSVTLRKKTKS
V294_S305del: Signal Peptide; Mature Protein:
SEQ ID NO: 24
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKS
K291_S305del: Signal Peptide; Mature Protein:
SEQ ID NO: 25
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
R285_S305del: Signal Peptide; Mature Protein:
SEQ ID NO: 26
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSS
S283_S305del: Signal Peptide; Mature Protein:
SEQ ID NO: 27
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQ
K298_S305del: Signal Peptide; Mature Protein:
SEQ ID NO: 51
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKSVTLR
R297_S305del: Signal Peptide; Mature Protein:
SEQ ID NO: 52
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKSVTL
S293_S305del: Signal Peptide; Mature Protein:
SEQ ID NO: 53
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KK
K292_S305del: Signal Peptide; Mature Protein:
SEQ ID NO: 54
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
K
SIGNAL PEPTIDES
Alpha mating factor (P01149):
SEQ ID NO: 28
MRFPSIFTAVLFAASSALAAPVNTTTEDETAQIPAEAVIGYSDLEGDFDVAVLPFSNS
TNNGLLFINTTIASIAAKEEGVS
Human Albumin Secretory Signal Peptide + Propeptide (P02768):
SEQ ID NO: 29
MKWVTFISLLFLFSSAYSRGVFRR
Human DNASE1L3 Signal Peptide (Q13609):
SEQ ID NO: 30
MSRELAPLLLLLLSIHSALA
LINKER SEQUENCES
SEQ ID NO: 31
GGGGS
SEQ ID NO: 32
GGGGSGGGGSGGGGS
SEQ ID NO: 33
APAPAPAPAPAPAP
SEQ ID NO: 34
AEAAAKEAAAKA
SEQ ID NO: 35
SGGSGSS
SEQ ID NO: 36
SGGSGGSGGSGGSGSS
SEQ ID NO: 37
SGGSGGSGGSGGSGGSGGSGGSGGSGGSGSS
SEQ ID NO: 38
GGSGGSGGSGGSGGSGGSGGSGGSGGSGS
OTHER SEQUENCES
Human Serum Albumin (Mature Protein):
SEQ ID NO: 39
DAHKSEVAHRFKDLGEENFKALVLIAFAQYLQQCPFEDHVKLVNEVTEFAKTCVADES
AENCDKSLHTLFGDKLCTVATLRETYGEMADCCAKQEPERNECFLQHKDDNPNLPRLV
RPEVDVMCTAFHDNEETFLKKYLYEIARRHPYFYAPELLFFAKRYKAAFTECCQAADK
AACLLPKLDELRDEGKASSAKQRLKCASLQKFGERAFKAWAVARLSQRFPKAEFAEVS
KLVTDLTKVHTECCHGDLLECADDRADLAKYICENQDSISSKLKECCEKPLLEKSHCI
AEVENDEMPADLPSLAADFVESKDVCKNYAEAKDVFLGMFLYEYARRHPDYSVVLLLR
LAKTYETTLEKCCAAADPHECYAKVFDEFKPLVEEPQNLIKQNCELFEQLGEYKFQNA
LLVRYTKKVPQVSTPTLVEVSRNLGKVGSKCCKHPEAKRMPCAEDYLSVVLNQLCVLH
EKTPVSDRVTKCCTESLVNRRPCFSALEVDETYVPKEFNAETFTFHADICTLSEKERQ
IKKQTALVELVKHKPKATKEQLKAVMDDFAAFVEKCCKADDKETCFAEEGKKLVAASQ
AALGL
Human Factor XI:
SEQ ID NO: 40
MIFLYQVVHFILFTSVSGECVTQLLKDTCFEGGDITTVFTPSAKYCQWCTYHPRCLL
FTFTAESPSEDPTRWFTCVLKDSVTETLPRVNRTAAISGYSFKQCSHQISACNKDIYV
DLDMKGINYNSSVAKSAQECQERCTDDVHCHFFTYATRQFPSLEHRNICLLKHTQTGT
PTRITKLDKVVSGFSLKSCALSNLACIRDIFPNTVFADSNIDSVMAPDAFVCGRICTH
HPGCLFFTFFSQEWPKESQRNLCLLKTSESGLPSTRIKKSKALSGFSLQSCRHSIPVF
CHSSFYHDTDFLGEELDIVAAKSHEACQKLCTNAVRCQFFTYTPAQASCNEGKGKCYL
KLSSNGSPTKILHGRGGISGYTLRLCKMDNECTTKIKPRIVGGTASVRGEWPWQVTLH
TTSPTQRHLCGGSIIGNQWILTAAHCFYGVESPKILRVYSGILNQSEIKEDTSFFGVQ
EIIIHDQYKMAESGYDIALLKLETTVNYTDSQRPICLPSKGDRNVIYTDCWVTGWGYR
KLRDKIQNTLQKAKIPLVTNEECQKRYRGHKITHKMICAGYREGGKDACKGDSGGPLS
CKHNEVWHLVGITSWGEGCAQRERPGVYTNWEYVDWILEKTQAV
Human prekallikrein:
SEQ ID NO: 41
MILFKQATYFISLFATVSCGCLTQLYENAFFRGGDVASMYTPNAQYCQMRCTFHPRCL
LFSFLPASSINDMEKRFGCFLKDSVTGTLPKVHRTGAVSGHSLKQCGHQISACHRDIY
KGVDMRGVNFNVSKVSSVEECQKRCTNNIRCQFFSYATQTFHKAEYRNNCLLKYSPGG
TPTAIKVLSNVESGFSLKPCALSEIGCHMNIFQHLAFSDVDVARVLTPDAFVCRTICT
YHPNCLFFTFYTNVWKIESQRNVCLLKTSESGTPSSSTPQENTISGYSLLTCKRTLPE
PCHSKIYPGVDFGGEELNVTFVKGVNVCQETCTKMIRCQFFTYSLLPEDCKEEKCKCF
LRLSMDGSPTRIAYGTQGSSGYSLRLCNTGDNSVCTTKTSTRIVGGTNSSWGEWPWQV
SLQVKLTAQRHLCGGSLIGHQWVLTAAHCFDGLPLQDVWRIYSGILNLSDITKDTPFS
QIKE111HQNYKVSEGNHDIALIKLQAPLNYTEFQKPICLPSKGDTSTIYTNCWVTGW
GFSKEKGEIQNILQKVNIPLVTNEECQKRYQDYKITQRMVCAGYKEGGKDACKGDSGG
PLVCKHNGMWRLVGITSWGEGCARREQPGVYTKVAEYMDWILEKTQSSDGKAQMQSPA
DNase epitope
SEQ ID NO: 42
MGDFNAGCSYV
ACTIVATABLE LINKER SEQUENCES
FXIIa-susceptible linker (Factor XI peptide):
SEQ ID NO: 43
CTTKIKPRIVGGTASVRGEWPWQVT
FXIIa-susceptible linker
SEQ ID NO: 44
GGGGSPRIGGGGS
FXIIa-susceptible linker (Prekallikrein peptide):
SEQ ID NO: 45
VCTTKTSTRIVGGTNSSWGEWPWQVS
FXIIa-susceptible linker (Prekallikrein peptide):
SEQ ID NO: 46
STRIVGG
BD-DELETED D1L3 FUSION PROTEINS
Albumin - DNASE1L3 Variant - Fusion Protein. (Albumin,
DNASE1L3 Variant):
SEQ ID NO: 47
DAHKSEVAHRFKDLGEENFKALVLIAFAQYLQQCPFEDHVKLVNEVTEFAKTCVADES
AENCDKSLHTLFGDKLCTVATLRETYGEMADCCAKQEPERNECFLQHKDDNPNLPRLV
RPEVDVMCTAFHDNEETFLKKYLYEIARRHPYFYAPELLFFAKRYKAAFTECCQAADK
AACLLPKLDELRDEGKASSAKQRLKCASLQKFGERAFKAWAVARLSQRFPKAEFAEVS
KLVTDLTKVHTECCHGDLLECADDRADLAKYICENQDSISSKLKECCEKPLLEKSHCI
AEVENDEMPADLPSLAADFVESKDVCKNYAEAKDVFLGMFLYEYARRHPDYSVVLLLR
LAKTYETTLEKCCAAADPHECYAKVFDEFKPLVEEPQNLIKQNCELFEQLGEYKFQNA
LLVRYTKKVPQVSTPTLVEVSRNLGKVGSKCCKHPEAKRMPCAEDYLSVVLNQLCVLH
EKTPVSDRVTKCCTESLVNRRPCFSALEVDETYVPKEFNAETFTFHADICTLSEKERQ
IKKQTALVELVKHKPKATKEQLKAVMDDFAAFVEKCCKADDKETCFAEEGKKLVAASQ
AALGLSGGSGGSGGSGGSGGSGGSGGSGGSGGSGSSMRICSFNVRSFGESKQEDKNAM
DVIVKVIKRCDIILVMEIKDSNNRICPILMEKLNRNSRRGITYNYVISSRLGRNTYKE
QYAFLYKEKLVSVKRSYHYHDYQDGDADVFSREPFVVWFQSPHTAVKDFVIIPLHTTP
ETSVKEIDELVEVYTDVKHRWKAENFIFMGDFNAGCSYVPKKAWKNIRLRTDPRFVWL
IGDQEDTTVKKSTNCAYDRIVLRGQEIVSSVVPKSNSVFDFQKAYKLTEEEALDVSDH
FPVEFKLQ
BD-Deleted D1L3 - Fc Fusion Protein. (Signal Peptide,
DNASE1L3, Fc Fragment):
SEQ ID NO: 50
MSRELAPLLLLLLSTHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSGGSGGS
GGSGGSGGSGGSGGSGGSGGSDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSVSVMHEA
LHNHYTQKSLSLSPGK
WILD-TYPE D1L3 FUSION PROTEINS
Albumin - WT DNASE1L3 - Fusion Protein. (Albumin. DNASE1L3):
SEQ ID NO: 48
DAHKSEVAHRFKDLGEENFKALVLIAFAQYLQQCPFEDHVKLVNEVTEFAKTCVADES
AENCDKSLHTLFGDKLCTVATLRETYGEMADCCAKQEPERNECFLQHKDDNPNLPRLV
RPEVDVMCTAFHDNEETFLKKYLYEIARRHPYFYAPELLFFAKRYKAAFTECCQAADK
AACLLPKLDELRDEGKASSAKQRLKCASLQKFGERAFKAWAVARLSQRFPKAEFAEVS
KLVTDLTKVHTECCHGDLLECADDRADLAKYICENQDSISSKLKECCEKPLLEKSHCI
AEVENDEMPADLPSLAADFVESKDVCKNYAEAKDVFLGMFLYEYARRHPDYSVVLLLR
LAKTYETTLEKCCAAADPHECYAKVFDEFKPLVEEPQNLIKQNCELFEQLGEYKFQNA
LLVRYTKKVPQVSTPTLVEVSRNLGKVGSKCCKHPEAKRMPCAEDYLSVVLNQLCVLH
EKTPVSDRVTKCCTESLVNRRPCFSALEVDETYVPKEFNAETFTFHADICTLSEKERQ
IKKQTALVELVKHKPKATKEQLKAVMDDFAAFVEKCCKADDKETCFAEEGKKLVAASQ
AALGLGGGGSGGGGSGGGGSMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKSVTLRKKTKSKRS
WT DNASE1L3 - Fc Fusion Protein. (Signal Peptide, DNASE1L3,
Fc Fragment):
SEQ ID NO: 49
MSRELAPLLLLLLSTHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSTNCA
YDRIVLRGQEIVSSVVPKSNSVFDFQKAYKLTEEEALDVSDHFPVEFKLQSSRAFTNS
KKSVTLRKKTKSKRSSGGSGGSGGSGGSGGSGGSGGSGGSGGSDKTHTCPPCPAPELL
GGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSK
LTVDKSRWQQGNVFSVSVMHEALHNHYTQKSLSLSPGK

What is claimed is:

1. A DNASE1-LIKE 3 (D1L3) enzyme comprising an amino acid sequence that has at least 70% sequence identity to amino acids 21 to 282 of SEQ ID NO: 4 or amino acids 21 to 252 of SEQ ID NO: 5, wherein the D1L3 enzyme has a deletion of at least the C-terminal serine residue, or at least three amino acids of the BD.

2. The D1L3 enzyme of claim 1, wherein the D1L3 enzyme has a deletion of at least five amino acids of the BD.

3. The D1L3 enzyme of claim 1, wherein the D1L3 enzyme has a deletion of at least eight amino acids of the BD.

4. The D1L3 enzyme of claim 1, wherein the D1L3 enzyme has a deletion of at least ten amino acids of the BD.

5. The D1L3 enzyme of claim 1, wherein the D1L3 enzyme has a deletion of at least twelve amino acids of the BD.

6. The D1L3 enzyme of claim 1, wherein the D1L3 enzyme has a deletion of at least fifteen amino acids of the BD.

7. The D1L3 enzyme of claim 1, wherein the D1L3 enzyme has a deletion of at least eighteen amino acids of the BD.

8. The D1L3 enzyme of claim 1, wherein the D1L3 enzyme has a deletion of at least twenty one amino acids of the BD.

9. The D1L3 enzyme of claim 1, wherein the D1L3 enzyme has a deletion of the full BD.

10. The D1L3 enzyme of any one of claims 1 to 9, wherein the amino acid deletions within the BD are independently selected from deletions at the N-terminus of the BD, the C-terminus of the BD, and internal to the BD.

11. The D1L3 enzyme of claim 10, wherein the BD deletions are taken from the C-terminus of the BD.

12. The D1L3 enzyme of claim 9, wherein the D1L3 enzyme has a deletion of an additional one to twenty amino acids from the C-terminal amino acids of SEQ ID NO:4 or SEQ ID NO:5.

13. The D1L3 enzyme of any one of claims 1 to 12, wherein the D1L3 enzyme comprises an amino acid sequence having at least 80% sequence identity to amino acids 21 to 282 of SEQ ID NO: 4 or amino acids 21 to 252 of SEQ ID NO: 5.

14. The D1L3 enzyme of claim 13, wherein the D1L3 enzyme comprises an amino acid sequence having at least 85% sequence identity to amino acids 21 to 282 of SEQ ID NO: 4 or amino acids 21 to 252 of SEQ ID NO: 5.

15. The D1L3 enzyme of claim 13, wherein the D1L3 enzyme comprises an amino acid sequence having at least 90% sequence identity to amino acids 21 to 282 of SEQ ID NO: 4 or amino acids 21 to 252 of SEQ ID NO: 5.

16. The D1L3 enzyme of claim 13, wherein the D1L3 enzyme comprises an amino acid sequence having at least 95% sequence identity to amino acids 21 to 282 of SEQ ID NO: 4 or amino acids 21 to 252 of SEQ ID NO: 5.

17. The D1L3 enzyme of any one of claims 1 to 16, comprising at least one building block substitution from D1 (SEQ ID NO:1), DNASE-1 LIKE 1 (D1L1) (SEQ ID NO:2), or DNASE-1-LIKE 2 (SEQ ID NO:3).

18. The D1L3 enzyme of any one of claims 1 to 17, wherein the D1L3 enzyme comprises 1 to 20 amino acid mutations selected from deletions and substitutions in the BD, optionally wherein the D1L3 enzyme comprises at least three, or at least five, or at least 10 amino acid substitutions or deletions.

19. The D1L3 enzyme of any one of claims 1 to 17, wherein the D1L3 enzyme is fused or conjugated to a carrier selected from a fusion partner or a polyethylene glycol (PEG).

20. The D1L3 enzyme of claim 19, wherein the fusion partner is albumin or Fe, which is optionally fused at the N-terminus and/or C-terminus.

21. The D1L3 enzyme of claim 20, wherein the D1L3 enzyme is fused to an albumin amino acid sequence at the N-terminal side of the mature enzyme, with a linking amino acid sequence between the albumin amino acid sequence and the amino acid sequence of the mature enzyme.

22. The D1L3 enzyme of claim 21, wherein the linker is a flexible or rigid linker, or comprises a protease cleavage site.

23. The D1L3 enzyme of claim 22, wherein the linker is from about 5 to about 50 amino acids in length.

24. The D1L3 enzyme of claim 23, wherein the linker is from about 10 to about 35 amino acids in length.

25. The D1L3 enzyme of claim 23, wherein the linker has from 15 to 35 amino acids.

26. The D1L3 enzyme of any one of claims 22 to 25, wherein the linker is a flexible linker.

27. The D1L3 enzyme of claim 26, wherein the amino acid sequence of the linker is predominately glycine and serine residues, or consists essentially of glycine and serine residues.

28. The D1L3 enzyme of claim 27, the ratio of Ser and Gly in the linker is from about 1:1 to about 1:10.

29. The D1L3 enzyme of claim 28, wherein the flexible linker comprises or consists essentially of an amino acid sequence of the formula (Gly_{y}Ser)ₙS_{z}, where y is from 1 to 10, n is from 1 to about 10, and z is 0 or 1.

30. The D1L3 enzyme of claim 29, wherein y is from 1 to 5.

31. The D1L3 enzyme of claim 29 or 30, wherein n is from 3 to 8.

32. The D1L3 enzyme of claim 29, wherein the linker comprises the amino acid sequence S(GGS)₄GSS (SEQ ID NO: 36), S(GGS)₉GSS (SEQ ID NO: 37), S(GGS)₉GSS (SEQ ID NO: 38).

33. The D1L3 enzyme of claim 22, wherein the linker is cleavable by a coagulation pathway protease.

34. The D1L3 enzyme of claim 33, wherein the linker is cleavable by Factor XII or a neutrophil protease.

35. The D1L3 enzyme of claim 1, wherein the D1L3 enzyme has the amino acid sequence of SEQ ID NO:47

36. An isolated polynucleotide encoding the D1L3 enzyme of any one of claims 1 to 35.

37. The isolated polynucleotide of claim 36, wherein the polynucleotide is an mRNA or a modified mRNA (mmRNA).

38. The isolated polynucleotide of claim 36, wherein the polynucleotide is DNA.

39. A vector for introducing the polynucleotide of any one of claims 36 to 38 to a host cell.

40. A host cell comprising the vector of claim 39.

41. A pharmaceutical composition comprising an effective amount of the D1L3 enzyme of any one of claims 1 to 35, the polynucleotide according to any one of claims 36 to 38, the vector according to claim 39, or the host cell according to claim 40, and a pharmaceutically acceptable carrier.

42. The pharmaceutical composition of claim 41, wherein the composition comprises an effective amount of the D1L3 enzyme of any one of claims 1 to 35, and a pharmaceutically acceptable carrier for parenteral administration.

43. The pharmaceutical composition of claim 41, formulated for topical, parenteral, or pulmonary administration.

44. The pharmaceutical composition of claim 43, formulated for intradermal, intramuscular, intraperitoneal, intraarticular, intravenous, subcutaneous, intraarterial, oral, sublingual, pulmonary, or transdermal administration.

45. A method for treating a subject in need of extracellular chromatin degradation, the method comprising administering the pharmaceutical composition of any one of claims 41 to 44 to a subject in need.

46. The method of claim 45, wherein the subject is in need of extracellular trap (ET) degradation and/or neutrophil extracellular trap (NET) degradation.

47. The method of claim 45 or 46, wherein the subject has a loss of function mutation in one or both D1L3 genes.

48. The method of any one of claims 45 to 46, wherein the subject has SLE.

49. The method of any one of claims 45 to 47, wherein the subject has a condition selected from chronic neutrophilia, neutrophil aggregation or leukostasis, thrombosis or vascular occlusion, ischemia-reperfusion injury, surgical or traumatic tissue injury, an acute or chronic inflammatory reaction or disease, an autoimmune disease, cardiovascular disease, metabolic disease, systemic inflammation, inflammatory disease of the respiratory tract, renal inflammatory disease, inflammatory disease related to transplanted tissue and cancer.

50. The method of any one of claims 45 to 47, wherein the subject has, or is at risk of, NETs occluding ductal systems, wherein the condition is optionally selected from pancreatitis, cholangitis, conjunctivitis, mastitis, dry eye disease, obstructions of vas deferens, and renal disease.

51. The method of any one of claims 45 to 47, wherein the subject has, or is at risk of, NETs accumulating on endothelial surfaces.

52. The method of claim any one of claims 45 to 51, wherein the D1L3 enzyme is parenterally administered to the subject approximately once or twice per week or once or twice per month.

53. A method for obtaining cell-free DNA from a subject, the method comprising:
administering a nuclease with chromatin-degrading activity;
obtaining a biological sample from the subject; and
isolating cell-free DNA from the sample.

54. The method of claim 53, wherein the biological sample is a biological fluid.

55. The method of claim 53, wherein the biological sample is selected from blood, serum, plasma.

56. The method of any one of claims 53 to 55, wherein the nuclease is selected from DNASE1-LIKE 3 (D1L3), DNASE1 (D1), DNASE1-LIKE 1 (D1L1), DNASE1-LIKE 2 (D1L2), DNASE1-LIKE 3 Isoform 2 (D1L3-2), DNASE2A (D2A), and DNASE2B (D2B) or a variant thereof.

57. The method of claim 56, wherein the nuclease is D1L3 having a deletion of at least 5 amino acids of the D1L3 basic domain.

58. The method of claim 56 or 57, wherein the nuclease has a fusion or conjugation to a half-life extension moiety.

59. The method of claim 58, wherein the half-life extension moiety is albumin.

60. The method of claim 59, wherein the nuclease is SEQ ID NO: 47.

61. The method of any one of claims 53 to 60, wherein the biological sample is isolated from the subject after about 10 minutes to about 1 day of administering the nuclease.

62. The method of claim 61, wherein the biological sample is isolated from the subject after about 10 minutes to about two hours, and optionally after about one hour of administering the nuclease.

63. The method of any one of claims 53 to 62, wherein the nuclease is administered at a dose of from about 0.001 mg/kg to about 100 mg/kg, optionally wherein the dose is from about 0.01 mg/kg to about 1 mg/kg.

64. The method of any one of claims 53 to 63, wherein the subject is suspected of having cancer, is at risk of having cancer, or is diagnosed as having cancer.

65. The method of claim 64, wherein the subject has previously had cancer, or has a genetic predisposition to develop cancer.

66. The method of any one of claims 53 to 65, wherein the method further comprises evaluating the cell-free DNA.

67. The method of claim 66, wherein the evaluating is performed by amplifying, hybridizing, and/or sequencing a genetic cancer marker; or by evaluating for an epigenetic profile.

68. The method of claim 66 or 67, wherein the genetic cancer marker is selected from a variant of one or more of the following genes that is associated with cancer: TP53, EGFR, CDKN2A, AKT1, JAK3, TSC1, NF1, CDH1, MML3, CTNNB1, PIK3C2G, GATA1, EPHB1, ESR1, PAK7, FLT4, MAP2K2, KRAS, NRAS, PIK3CA, BRAF, SMAD4, and APC.

69. The method of any one of claims 66 to 68, wherein the evaluating comprises one or more of assessment of tumor mutations, single nucleotide polymorphism (SNPs), microsatellite instability, copy number aberrations (e.g. relative to a reference genome, or parental genome) or cancer-specific or cancer-associated changes (e.g., methylation signature changes).

70. The method of any one of claims 66 to 68, wherein the evaluating comprises detecting histone modifications.

71. The method of any one of claims 66 to 70, wherein the cell free DNA is evaluated for markers of one or more of: colorectal cancer, bladder cancer, brain cancer, breast cancer, pancreatic cancer, liver cancer, endometrial cancer, gastroesophageal cancer, head and neck cancer, hepatocellular cancer, lung cancer (e.g. non-small cell lung cancer and small cell lung cancer), melanoma, bone cancer, ovarian cancer, testicular cancer, prostate cancer, renal cancer, lymphoma, thyroid cancer, and hematological malignancy.

72. A method for treating a subject in need of extracellular chromatin degradation, the method comprising administering a polynucleotide comprising a nucleotide sequence encoding DNASE1L3 or a variant thereof, or administering cells comprising the polynucleotide.

73. The method of claim 72, the method comprising: transforming a cell in vitro with an exogenous polynucleotide comprising a nucleotide sequence encoding a DNASE1L3 or a variant thereof, optionally wherein the cell is obtained from the subject; optionally culturing, growing and/or expanding the cell in vitro to generate a progeny of the cell;
and administering the cell or the progeny of the cell to the subject.

74. The method of claim 72, wherein the polynucleotide is DNA.

75. The method of claim 74, wherein the DNA is a vector or an expression vector.

76. The method of claim 75, wherein the vector or the expression vector is an adeno-associated viral vector (AAV).

77. The method of claim 72 or claim 73, wherein the polynucleotide is an mRNA.

78. The method of claim 77, wherein the mRNA is a modified mRNA (mmRNA).

79. The method of any one of claims 72 to 78, wherein cells transformed with the polynucleotide secrete D1L3 enzyme.

80. The method of claim 79, wherein the polynucleotide encodes an enzyme comprising the wild-type D1L3 amino acid sequence.

81. The method of claim 79, wherein the polynucleotide encodes an enzyme comprising a D1L3 having a deletion of the Basic Domain.

82. The method of claim 80 or 81, wherein the secreted D1L3 enzyme has a deletion in the C-terminal Basic Domain.

83. The method of claim 82, wherein the D1L3 enzyme has a deletion of at least one amino acid, or at least 3, or at least 5, or at least 8, or at least 9, or at least 13, or at least 14 or at least 15 C-terminal amino acids of the D1L3 basic domain.

84. The method of claim 83, wherein the deletion retains each of K291, K292, R297, K298, K299, K303, and R304 with respect to SEQ ID NO: 4.

85. The method of claim 83, wherein the deletion removes K303 and/or R304 with respect to SEQ ID NO: 4, and wherein the deletion retains K291, K292 and R297, K298 and K299 with respect to SEQ ID NO: 4.

86. The method of claim 83, wherein the deletion removes K303 and R304, and one or more of R297, K298 and K299 with respect to SEQ ID NO: 4, and wherein the deletion retains K291 and K292 with respect to SEQ ID NO: 4.

87. The method of claim 83, wherein the deletion removes K303, R304, R297, K298 and K299 with respect to SEQ ID NO: 4, and wherein the deletion retains one or both of K291 and/or K292 with respect to SEQ ID NO: 4.

88. The method of claim 83, wherein the deletion removes each of K291, K292, R297, K298, K299, K303, and R304 with respect to SEQ ID NO: 4.

89. The method of any one of claims 72 to 88, wherein the D1L3 has a fusion to a carrier protein.

90. The method of claim 89, wherein the carrier protein is an albumin or an Fc domain.

91. The method of claim 90, wherein the D1L3 fusion contains a linker sequence, which is optionally a flexible linker.

92. The method of claim 88, wherein the nuclease is SEQ ID NO: 47.

93. The method of any one of claims 72 to 92, wherein the polynucleotide is administered to the subject.

94. The method of any one of claims 72 to 92, wherein the polynucleotide is expressed in a cell and administered to the subject.

95. The method of claim 94, wherein the cell is a hematopoietic cell.

96. The method of claim 95, wherein the cell is a T cell, a B cell, macrophage, dendritic cell, or a stem cell.

97. The method of claim 96, wherein the cells comprise CD8+ and/or CD4+ T cells.

98. The method of any one of claims 72 to 97, wherein the subject has cancer or a viral infection.

99. The method of claim 98, wherein the composition is administered with cancer therapy, or the cell is a CAR-T cell, or the cell is a T cell specific for a tumor antigen.
